(19) United States
(12) Patent Application Publication
(10) Pub. No.: US 2024/0158466 A1
(43) Pub. Date: May 16, 2024

(54) PEGYLATED P-SELECTIN INHIBITORS

(71) Applicant: Beth Israel Deaconess Medical Center, Inc., Boston, MA (US)

(72) Inventors: Elliot Chaikof, Newton, MA (US); Richard D. Cummings, Brookline, MA (US)

(73) Assignee: Beth Israel Deaconess Medical Center, Inc., Boston, MA (US)

(21) Appl. No.: 18/272,092

(22) PCT Filed: Jan. 13, 2022
(86) PCT No.: PCT/US2022/012253

§ 371 Date: Jul. 12, 2023

Related U.S. Application Data
(60) Provisional application No. 63/137,354, filed on Jan. 14, 2021.

(51) Int. Cl. C07K 14/705; A61K 47/54; A61K 47/60; A61P 7/02; C07K 1/107

(52) U.S. Cl. CPC C07K 14/70564; A61K 47/549; A61K 47/60; A61P 7/02; C07K 1/1077

(57) ABSTRACT

This disclosure relates to PEGylated selectin inhibitors, compositions, and methods related thereto. In certain embodiments, the disclosure relates to glycopeptides that contain one or more modified amino acids conjugated to a saccharide or polysaccharide and a polyethylene glycol (PEG) moiety. In some embodiments, the disclosure relates to uses of the PEGylated glycopeptides as anti-inflammatory, anti-thrombotic, or anti-metastatic agents.

### CROSS REFERENCE TO RELATED APPLICATIONS

This application claims priority under 35 U.S.C. § 119(e) to U.S. Provisional Application No. 63/137,354, filed Jan. 14, 2021, the contents of which is incorporated herein by reference in its entirety.

### GOVERNMENT SUPPORT

This invention was made with government support under Grant No. HL128237, DK107405, and GM116196, awarded by the National Institutes of Health. The government has certain rights in the invention.

### BACKGROUND

As many as one million people in the United States are affected each year by venous thromboembolism (VTE), with significant associated morbidity and mortality.^{1,2} Pulmonary embolus (PE) and deep venous thrombosis (DVT) constitute the majority of venous thrombotic events and necessitate systemic anticoagulation to prevent clot propagation and fatal PE.³ However, all anticoagulants increase the risk of bleeding with hemorrhagic complications occurring in up to 10% of individuals during the initial course of treatment depending on the drug regimen, indication, and duration of therapy.⁴⁻⁶ Despite the initial promise that direct thrombin and factor Xa inhibitors would display superior safety profiles compared to warfarin, the risk of bleeding in “real-world” settings remains in the range of 5 to 10% or higher.^{6,7} As such, there is an urgent need for safer therapies.

It is now well recognized that the pathophysiology of VTE extends beyond Virchow's triad of venous stasis, hypercoagulability, and endothelial dysfunction.⁸ Increased rates of VTE in patients with chronic inflammatory conditions, such as inflammatory bowel disease or rheumatoid arthritis highlight that a systemic inflammatory state is an important risk factor for VTE.^{9,10} Mechanistic studies have confirmed that venous thrombi contain not only neutrophils, monocytes, and platelet-leukocyte aggregates, but have revealed their critical role in venous thrombus formation and propagation.¹¹ Numerous studies have demonstrated that monocyte- and neutrophil-derived tissue factor (TF) is a decisive initial trigger for fibrin deposition,^{12,13} as well as the role of neutrophil extracellular traps in promoting thrombus propagation.¹⁴⁻¹⁸ In principle, targeting leukocyte driven events rather than the coagulation cascade alone may provide a means to prevent or treatment of venous thrombosis while decreasing bleeding risk.

### SUMMARY OF THE INVENTION

The present disclosure provides isolated, non-naturally occurring PEGylated glycopeptides, compositions, and methods related thereto. In certain embodiments, the PEGylated glycopeptides, salts thereof, or compositions thereof provided herein inhibit P-selectin with similar potency to the corresponding non-PEGylated glycopeptide. The present disclosure also provides methods of using the PEGylated glycopeptides for treating or preventing cardiovascular disease, lung disease, or allergy in a subject. In certain embodiments, the present disclosure provides methods of using compounds and compositions provided herein for inhibiting P-selectin in a cell of a subject or biological sample.

In one aspect, the disclosure provides glycopeptides comprising Y¹X¹Y²X²X³Y³X⁴X⁵X⁶Z¹X⁷W¹ (SEQ ID NO: 1), or salts thereof, wherein:
W¹ is threonine or serine substituted with a saccharide or polysaccharide,
X¹, X², X³, X⁴, X⁵, X⁶, and X⁷ are each individually and independently any amino acid,
Y¹, Y², and Y³ are each individually and independently tyrosine, phenylalanine, or phenylglycine, and wherein Y¹, Y², and Y³ are each independently unsubstituted or substituted with —SO₃H, —CH₂SO₃H, —CF₂SO₃H, —CO₂H, —CONH₂, —NHSO₂CH₃, —SO₂NH₂, or —CH₂PO₃H;
wherein at least one of Y¹, Y², and Y³ is substituted with —CH₂SO₃H:
Z¹ is proline or hydroxyproline; and
at least one amino acid residue is substituted with -L¹-PEG:
wherein L¹ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, or combinations thereof; and
PEG is linear or branched polyethylene glycol.

In certain embodiments, the glycopeptide, or a salt thereof, comprises the formula KEY¹EY²LDY³DFLZ¹EW¹EPL (SEQ ID NO: 12), wherein:
W¹ is threonine substituted with a polysaccharide,
Y¹, Y², and Y³ are phenylalanine 4-substituted with —CH₂SO₃H,
Z¹ is proline,
the polysaccharide comprises 2-(acetylamino)-2-deoxy-galactose alpha 1 bonded to W¹, a first galactose beta 3 bonded to 2-(acetylamino)-2-deoxy-galactose, 2-(acetylamino)-2-deoxy-glucose beta 6 bonded to 2-(acetylamino)-2-deoxy-galactose, fucose alpha 3 bonded to 2-(acetylamino)-2-deoxyglucose, a second galactose beta 4 bonded to 2-(acetylamino)-2-deoxy-glucose, and 5-acetamido-3,5-dideoxy-glycero-galacto-2-nonulosonic acid alpha 3 bonded to the first galactose,
K is substituted with -L¹-PEG, wherein
the PEG is linear and terminates in —OCH₃: and
L¹ has the structure

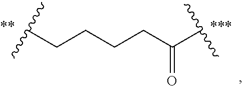 [SMILES: *CCCCC(*)=O]

wherein “**” is attached to PEG, and “***” is attached to K.

In certain embodiments, the glycopeptide, or a salt thereof, comprises the formula KEY¹EY²LDY³DFLZ¹EW¹EPL (SEQ ID NO: 12), wherein:
W¹ is threonine substituted with a polysaccharide,
Y¹, Y², and Y³ are phenylalanine 4-substituted with —CH₂SO₃H,
Z¹ is proline,
the polysaccharide comprises 2-(acetylamino)-2-deoxy-galactose alpha 1 bonded to W¹, a first galactose beta 3 bonded to 2-(acetylamino)-2-deoxy-galactose, 2-(acetylamino)-2-deoxy-glucose beta 6 bonded to 2-(acetylamino)-2-deoxy-galactose, fucose alpha 3 bonded to 2-(acetylamino)-2-deoxyglucose, a second galactose beta 4 bonded to 2-(acetylamino)-2-deoxy-glucose, and 5-acetamido-3,5-dideoxy-glycero-galacto-2-nonulosonic acid alpha 3 bonded to the first galactose,
K is substituted with -L¹-PEG, wherein
the PEG is branched and terminates in —OCH₃; and
L¹ has the structure

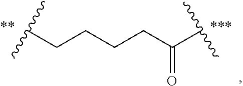 [SMILES: *CCCCC(*)=O]

wherein “**” is attached to PEG, and “***” is attached to K.

In certain embodiments, the glycopeptide, or salt thereof, is characterized as having similar efficacy to that of the corresponding non-PEGylated glycopeptide. In some embodiments, the glycopeptide, or salt thereof, is characterized as having an IC₅₀ for P-selectin similar to that of the corresponding non-PEGylated glycopeptide.

In another aspect, the present disclosure provides pharmaceutical compositions comprising an effective amount of a glycopeptide provided herein (e.g., a glycopeptide of SEQ ID NO: 1), or a salt thereof, and optionally a pharmaceutically acceptable excipient.

In another aspect, the present disclosure provides methods of treating or preventing cardiovascular disease, atherosclerosis, atherosclerotic lesions, thrombus formation, thromboembolism, stroke, myocardial infarction, or cancer in a subject comprising administering to the subject an effective amount of a glycopeptide provided herein (e.g., a glycopeptide of SEQ ID NO: 1), or a salt thereof, or a pharmaceutical composition thereof. In certain embodiments, the subject is at risk of, exhibiting symptoms of, or diagnosed with atherosclerosis, atherosclerotic lesions, thrombus formation, thromboembolism, stroke, myocardial infarction, or cancer.

In another aspect, the present disclosure provides methods of treating or preventing allergy or lung diseases in a subject comprising administering to the subject an effective amount of a glycopeptide provide herein (e.g., a glycopeptide of SEQ ID NO: 1), or a salt thereof, or a pharmaceutical composition thereof. In certain embodiments, the subject is at risk of, exhibiting symptoms of, or diagnosed with asthma, bronchitis, emphysema, and chronic obstructive pulmonary disease (COPD).

In yet another aspect, the present disclosure provides methods of inhibiting P-selectin comprising contacting a cell, tissue, or biological sample with an effective amount of a glycopeptide provided herein (e.g., a glycopeptide of SEQ ID NO: 1), or a salt thereof, or a pharmaceutical composition thereof.

The details of certain embodiments of the invention are set forth in the Detailed Description of Certain Embodiments, as described below. Other features, objects, and advantages of the invention will be apparent from the Definitions, Examples, Figures, and Claims. It should be understood that the aspects described herein are not limited to specific embodiments, methods, or configurations, and as such can, of course, vary. It is also to be understood that the terminology used herein is for the purpose of describing particular aspects only and, unless specifically defined herein, is not intended to be limiting.

### BRIEF DESCRIPTION OF THE DRAWINGS

The accompanying drawings, which are incorporated in and constitute a part of this specification, illustrate several embodiments of the invention and together with the description, provide non-limiting examples of the invention.

FIGS. 1A-B show the synthesis of PEG40-GSnP-6 (PG-6). P-selectin antagonist GSnP-6 is modeled after the N-terminus of human PSGL-1, including sialyl Lewis^{X} (N-acetylneuraminic acid) containing hexasaccharide and sulfopeptide epitopes responsible for high affinity binding to P-selectin. A linear 40 kDa monomethoxy-PEG-succinimidyl valerate (mPEG-SVA) was conjugated to the ε-amino residue of the N-terminal lysine of GSnP-6 affording PG-6. The glycan short form of PG-6 is shown in FIG. 1A, and the full chemical structure of PG-6 is shown in FIG. 1B.

FIGS. 2A-D show quantification of PG-6 in plasma. FIGS. 2A-C show liquid chromatography tandem mass spectrometry (LC-MS/MS) used to generate a standard curve of PG-6 concentration in blood plasma. FIG. 2A summarizes LC-MS/MS parameters for in-source fragmentation and rigor of the calibration curve. FIG. 2B shows generation of the quantifying peptide. FIG. 2C shows a stepped higher-energy collisional dissociation approach effectively fragmented PG-6 in a single MS/MS spectrum. Fragment ions: b-ions, from the N-terminus: y-ions, from the C-terminus: * ions, glycan loss. Modified amino acids: bold faced. FIG. 2D shows plasma concentration of PG-6 over time after intravenous administration of a single weight-based (8 μmol/kg) dose.

FIGS. 3A-D show GSnP-6 and PG-6 inhibit P-selectin binding to leukocytes. FIG. 3A shows GSnP-6 (0-100 μM) incubated with mouse neutrophils and monocytes. FIG. 3B shows PG-6 (0-100 μM) incubated with mouse neutrophils and monocytes. FIG. 3C shows GSnP-6 (0-100 μM) incubated with human neutrophils and monocytes. FIG. 3D shows PG-6 (0-100 μM) incubated with human neutrophils and monocytes. Flow cytometry was used to evaluate the percent binding inhibition of species appropriate P-selectin chimera to neutrophils or monocytes produced by GSnP-6 and PG-6 as compared to phosphate buffered saline control. Both GSnP-6 and PG-6 inhibit P-selectin leukocyte interactions in a dose-dependent manner. Data are represented as mean±SEM, n=3/agent/study.

FIGS. 4A-F show PG-6 inhibition of platelet-leukocyte aggregation in vitro and in vivo. FIGS. 4A-B show inhibition of platelet-leukocyte aggregation in vitro. Anticoagulated mouse or human blood was dosed with 120 μM PG-6 or saline control. Platelet-leukocyte aggregation was induced by adding species-specific PAR peptide and samples were analyzed using flow cytometry to quantify platelet-positive monocytes or neutrophils. FIG. 4A shows PG-6 significantly reduced platelet-leukocyte aggregation in mouse blood. FIG. 4B shows PG-6 significantly reduced platelet-leukocyte aggregation in human blood. Unstimulated control blood is included for reference. FIGS. 4C-F show inhibition of platelet-leukocyte aggregation in vivo. Intravital microscopy was used to characterize TNF-α-induced venular inflammation. Immediately prior to cremaster exteriorization, mice were infused with PG-6 or saline vehicle control and anti-platelet CD42b-Dylight 649 (red)/anti-neutrophil Alexa Fluor 488 (green). Platelet fluorescent signal was normalized to vessel area. FIG. 4C reports normalized platelet fluorescence as median integrated fluorescence over time. FIG. 4D reports normalized platelet fluorescence as area under the curve (AUC). FIG. 4E shows PG-6 significantly reduced platelet accumulation to adherent neutrophils. FIG. 4F shows platelet accumulation in mice administered saline vehicle. Data are mean±SEM, ***p<0.001, *p<0.05 (Student's t-test).

FIGS. 5A-F show treatment with PG-6 reduces venous thrombus formation. A non-occlusive thrombus was induced by electrolytic injury of the inferior vena cava and vessel thrombus weight measured 48 h after injury to determine treatment efficacy. FIG. 5A shows prophylactic administration of PG-6 (8 μmol/kg IV) and LMWH (6 mg/kg SC) demonstrated significant reduction in thrombus weight as compared to mice administered saline vehicle (n=8/group). FIG. 5B shows representative images of excised infrarenal vena cava 48 h after electrolytic injury. FIG. 5C shows quantification of Ly6G+ neutrophils. FIG. 5D shows quantification of CD68+ macrophages. Mice administered PG-6 demonstrate significantly less wall inflammation as compared to mice administered saline vehicle. FIG. 5E shows neutrophil infiltration within the vein wall 48 h after thrombus induction. FIG. 5F shows macrophage infiltration within the vein wall 48 h after thrombus induction. Arrowheads indicate positive immunostaining, (L) indicates lumen. Data are mean±SEM; ***p<0.001 (ANOVA with Tukey's multiple comparison).

FIG. 6 shows treatment with PG-6 does not affect hemostasis. The effect of PG-6 on hemostasis was assessed using a tail vein bleeding assay. Mice were subject to intravenous administration of saline vehicle, PG-6 (8 μmol/kg) or LMWH (6 mg/kg) 5 min prior to transection of the lateral tail vein. LMWH demonstrated a significant increase in bleeding time as compared to mice receiving saline vehicle while no increase in bleeding time was observed after administration of PG-6. Data is represented as mean±SEM. Group comparisons were conducted using Welch's ANOVA with Dunnett's multiple comparison test. ***p<0.001.

FIG. 7 shows the MALDI profile of PG-6. Data was collected in reflector negative ion mode with CHCA matrix.

FIG. 8 shows the reverse phase HPLC profile of PG-6.

FIGS. 9A-C show the extracted ion chromatograms of the quantifying peptide from plasma of mice. FIG. 9A shows the extracted ion chromatograms of the quantifying peptide from plasma of mice that were administered PG-6. FIG. 9B shows the extracted ion chromatograms of the quantifying peptide from plasma of mice that were administered saline.

FIG. 9C shows the extracted ion chromatograms of the quantifying peptide from plasma of mice that received no injection. The spectrum list at retention time 18.68 min from mass range 12(X)-1204 m/z are listed below the chromatograms. The quantifying peptide was only observed in mice dosed with PG-6. Chromatograms displayed in FIGS. 9B-C are plotted at an increased signal magnitude.

FIG. 10 shows PG-6 inhibition of platelet-leukocyte aggregation in vitro. Anticoagulated mouse or human blood was dosed with saline or PG-6 (120 μM) and platelets were activated with PAR peptide to examine platelet-leukocyte aggregation. (Top row) resting platelets displayed for reference; (middle row) saline-treated, PAR activated; (bottom row) PG-6-treated, PAR activated. Samples were stained with species specific CD45 and anti-CD41-PE (mouse platelets) or anti-CD42a-PE (human platelets), monocytes and neutrophils were discerned by characteristic forward/side scatter. Representative scatter plots shown, gated box indicates % platelet-leukocyte aggregates (PLA+%).

### DEFINITIONS

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3_{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts: or preferred isomers can be prepared by asymmetric syntheses. See, for example, Jacques et al., Enantiomers, Racenates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962): and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

When a range of values is listed, it is intended to encompass each value and sub-range within the range. A range is inclusive of the values at the two ends of the range unless otherwise provided. For example “C₁₋₆ alkyl” encompasses, C₁, C₂, C₃, C₄, C₅, C %, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, see-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the halo alkyl moiety has 1 to 8 carbon atoms (“C₁₋₈ haloalkyl”). In some embodiments, the haloalkyl moiety has 1 to 7 carbon atoms (“C₁₋₇ haloalkyl”).In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g. inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”).

In some embodiments, a heteroalkyl group is a saturated group having 1 to 4 carbon atoms and 1 or 2 heteroatoms within the parent chain (“heteroC₁₋₄alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₁ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₂ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₅), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

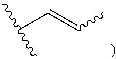

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g. inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”).

In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂) alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“CIA alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of Cia alkynyl groups include, without limitation, methylidenyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃0.14 carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₄₋₆ carbocyclyl”). In some embodiments, a carbocyclyl group has 5 to 10 ring carbon atoms (“C₃₋₆ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆, cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction.

Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}—, —ON(R^{bb})₂, —N(R^{bb})₂, —N(R^{bb})₃⁺X⁻, —N(OR^{cc})R^{bb}, —SH, —SR^{aa}—, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{cc}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd}substituents are joined to form ═O or ═S; wherein X⁻ is a counterion:
each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and
each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁ to alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxy,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1}is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono-or di-heteroaliphaticamino, mono- or di-alkylamino, mono- or di-heteroalkylamino, mono- or di-arylamino, or mono- or di-heteroarylamino; or two R^{X1}groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom: and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, n-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1, 1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), s -trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc. Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom: and each R^{bb} is independently hydrogen, substituted (e.g., substituted with one or more halogen) or unsubstituted C₁₋₁₀ alkyl, or an oxygen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁺, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methoxy, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3′″-[N-(imidazolylmethyl)]trityl Ether (IDTr-OR), 4,4′-Dimethoxy-3′″-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 1(0, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

A “leaving group” (LG) is an art-understood term referring to an atomic or molecular fragment that departs with a pair of electrons in heterolytic bond cleavage, wherein the molecular fragment is an anion or neutral molecule. As used herein, a leaving group can be an atom or a group capable of being displaced by a nucleophile. See e.g., Smith, March Advanced Organic Chemistry 6th ed. (501-502). Exemplary leaving groups include, but are not limited to, halo (e.g., fluoro, chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Additional examples of suitable leaving groups include, but are not limited to, halogen alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some embodiments, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some embodiments, the leaving group is a brosylate, such as p-bromobenzenesulfonyloxy. In some embodiments, the leaving group is a nosylate, such as 2-nitrobenzenesulfonyloxy. In some embodiments, the leaving group is a sulfonate-containing group. In some embodiments, the leaving group is a tosylate group. In some embodiments, the leaving group is a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

The term “glycopeptide” refers to a compound in which one or more carbohydrate moieties (e.g., a saccharide or polysaccharide) are covalently attached to a peptide or oligopeptide. Carbohydrates may be attached through the side chains of the amino acid residues that constitute the peptide or oligopeptide. Typically, carbohydrates are attached through amino acid side chains that comprise hydroxyl, amino, amide, or carboxyl groups.

The term “saccharide” refers to any of a class of carbohydrates that cannot be broken down to simpler sugars by hydrolysis and that comprise the building blocks of oligosaccharides and polysaccharides. Saccharides can occur as chains or rings and may be in the D- or L-configuration. Saccharides consist of at least three carbon atoms, one of which is attached to an oxygen atom (e.g., to form an aldehyde group (CHO) or a ketone in the open-chain form), and the others of which are each attached to a hydroxyl group (OH). Saccharides include, but are not limited to glucose, neuraminic acid, galactose, fucose, ribose, mannose, and fructose. Saccharides and the like may be further substituted.

The term “polysaccharide” refers to carbohydrates comprising multiple saccharide units bound together by glycosidic linkages. Polysaccharides include disaccharides, oligosaccharides, and polymeric polysaccharides. Polysaccharides may have linear or branched structures. The constituent units of a polysaccharide may be the same saccharide or distinct saccharides. Polysaccharides include, but are not limited to, disaccharides, such as sucrose, lactose, maltose, cellobiose, trehalose, and melibiose. Polysaccharides include, but are not limited to, oligosaccharides such as raffinose, stachyose, amylose, Lewis X. Lewis Y, Lewis A, Lewis B, sialyl Lewis X, and sialyl Lewis A. Polymeric polysaccharides include, but are not limited to, starch, glycogen, cellulose, chitin, xylan, arabinoxylan, mannan, fucoidan, galactomannan, callose, laminarin, chrysolaminarin, amylopectin, dextran, dextrins, maltodextrins, inulin, oligofructose, and polydextrose. Polysaccharides and the like may be further substituted.

The term “polyethylene glycol” or “PEG” refers to a polymer comprising ethylene glycol monomer units of the formula —[CH₂CH₂O]ₙ— or —[OCH₂CH₂]ₙ—, wherein n designates the number of repeat units. Typically, the polyethylene glycol contains about 20 to about 2,000,000 repeat units, typically about 20 to about 3,500 repeat units. The polyethylene glycol may be linear or branched. Linear polyethylene glycol is polyethylene glycol in which the repeat units are connected in a linear fashion, that is, in a straight chain. Branched polyethylene glycol is polyethylene glycol comprising at least one branching point or branching moiety through which three or more repeat units are covalently connected. In certain embodiments, a compound having a branched PEG comprises -L¹-(PEG)ₓ, wherein L¹ is substituted with x instances of PEG and x is an integer from 3 to 5,000. In some embodiments, a compound having a branched PEG comprises -L¹-(PEG)ₓ, wherein L¹ is substituted with x instances of PEG and x is an integer from 3 to 500. In certain embodiments, a compound having a branched PEG comprises -L¹-(PEG)ₓ, wherein L¹ is substituted with x instances of PEG and x is an integer from 3 to 100. In some embodiments, a compound having a branched PEG comprises -L¹-(PEG)ₓ, wherein L¹ is substituted with x instances of PEG and x is an integer from 3 to 20. The polyethylene glycol or PEG may terminate in a hydroxy group (—OH), substituted hydroxyl, or an oxygen atom substituent.

The disclosure is not intended to be limited in any manner by the above exemplary listing of substituents. Additional terms may be defined in other sections of this disclosure.

### Other Definitions

The following definitions are more general terms used throughout the present application.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N′(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example. Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “target tissue” refers to any biological tissue of a subject (including a group of cells, a body part, or an organ) or a part thereof, including blood and/or lymph vessels, which is the object to which a compound, particle, and/or composition of the disclosure is delivered. A target tissue may be an abnormal or unhealthy tissue, which may need to be treated. A target tissue may also be a normal or healthy tissue that is under a higher than normal risk of becoming abnormal or unhealthy, which may need to be prevented. In certain embodiments, the target tissue is the liver. In certain embodiments, the target tissue is the lung. A “non-target tissue” is any biological tissue of a subject (including a group of cells, a body part, or an organ) or a part thereof, including blood and/or lymph vessels, which is not a target tissue.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the disclosure may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for inhibiting P-selectin in a subject. In certain embodiments, a therapeutically effective amount is an amount sufficient for treating a disease (e.g., cardiovascular disease, atherosclerosis, atherosclerotic lesions, thrombus formation, thromboembolism, stroke, myocardial infarction, allergy, lung disease, asthma, bronchitis, emphysema, and chronic obstructive pulmonary disease (COPD)) in a subject. In certain embodiments, a therapeutically effective amount is an amount sufficient for inhibiting P-selectin and treating a disease (e.g., cardiovascular disease, atherosclerosis, atherosclerotic lesions, thrombus formation, thromboembolism, stroke, myocardial infarction, allergy, lung disease, asthma, bronchitis, emphysema, and chronic obstructive pulmonary disease (COPD)) in a subject.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for inhibiting P-selectin in a subject. In certain embodiments, a prophylactically effective amount is an amount sufficient for preventing a disease (e.g., cardiovascular disease, atherosclerosis, atherosclerotic lesions, thrombus formation, thromboembolism, stroke, myocardial infarction, allergy, lung disease, asthma, bronchitis, emphysema, and chronic obstructive pulmonary disease (COPD)) in a subject. In certain embodiments, a prophylactically effective amount is an amount sufficient for inhibiting P-selectin and preventing a disease (e.g., cardiovascular disease, atherosclerosis, atherosclerotic lesions, thrombus formation, thromboembolism, stroke, myocardial infarction, allergy, lung disease, asthma, bronchitis, emphysema, and chronic obstructive pulmonary disease (COPD)) in a subject.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

The term “cardiovascular disease” refers to diseases and disorders of the heart and circulatory system. Exemplary cardiovascular diseases, including cholesterol- or lipid-related disorders, include, but are not limited to acute coronary syndrome, angina, arrhythmia, arteriosclerosis, atherosclerosis, carotid atherosclerosis, cerebrovascular disease, cerebral infarction, congestive heart failure, congenital heart disease, coronary heart disease, coronary artery disease, coronary plaque stabilization, dyslipidemias, dyslipoproteinemias, endothelium dysfunctions, familial hypercholeasterolemia, familial combined hyperlipidemia, hypoalphalipoproteinemia, hypertriglyceridemia, hyperbetalipoproteinemia, hypercholesterolemia, hypertension, hyperlipidemia, intermittent claudication, ischemia, ischemia reperfusion injury, ischemic heart diseases, cardiac ischemia, metabolic syndrome, multi-infarct dementia, myocardial infarction, obesity, peripheral vascular disease, reperfusion injury, restenosis, renal artery atherosclerosis, rheumatic heart disease, stroke, thrombotic disorder, transitory ischemic attacks, and lipoprotein abnormalities associated with Alzheimer's disease, obesity, diabetes mellitus, syndrome X, impotence, multiple sclerosis, Parkinson's diseases and inflammatory diseases.

The term “allergy” refers to the condition of immune hypersensitivity that is greater than normal in an individual who has been exposed to an allergen and has responded with an overproduction of certain immune system components such as immunoglobulin E (IgE) antibodies. Exemplary allergic conditions include, but are not limited to, asthma, eczema, allergic rhinitis or coryza, hay fever, conjunctivitis, bronchial asthma, urticaria (hives) and food allergies, reactions to the venom of stinging insects such as wasps and bees, as well as other atopic conditions such as atopic dermatitis, anaphylaxis, drug allergy, angioedema, and allergic conjunctivitis.

The term “lung disease” or “pulmonary disease” refers to a disease of the lung. Examples of lung diseases include, but are not limited to, bronchiectasis, bronchitis, bronchopulmonary dysplasia, interstitial lung disease, occupational lung disease, emphysema, cystic fibrosis, acute respiratory distress syndrome (ARDS), severe acute respiratory syndrome (SARS), asthma (e.g., intermittent asthma, mild persistent asthma, moderate persistent asthma, severe persistent asthma), chronic bronchitis, chronic obstructive pulmonary disease (COPD), emphysema, interstitial lung disease, sarcoidosis, asbestosis, aspergilloma, aspergillosis, pneumonia (e.g., lobar pneumonia, multilobar pneumonia, bronchial pneumonia, interstitial pneumonia), pulmonary fibrosis, pulmonary tuberculosis, rheumatoid lung disease, pulmonary embolism, and lung cancer (e.g., non-small-cell lung carcinoma (e.g., adenocarcinoma, squamous-cell lung carcinoma, large-cell lung carcinoma), small-cell lung carcinoma).

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast): brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer: epithelial carcinoma; ependymoma; endotheliosarcoma (e.g., Kaposi's sarcoma, multiple idiopathic hemorrhagic sarcoma): endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST): germ cell cancer: head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLUSLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma): liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung): leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)): small bowel cancer (e.g., appendix cancer): soft tissue sarcoma (e.g., malignant fibrous histiocytoma (MFH), liposarcoma, malignant peripheral nerve sheath tumor (MPNST), chondrosarcoma, fibrosarcoma, myxosarcoma): sebaceous gland carcinoma; small intestine cancer: sweat gland carcinoma: synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

As used herein, the terms “inhibition,” “inhibiting,” “inhibit,” or “inhibitor” refer to the ability of a compound or glycopeptide to reduce, slow, halt or prevent activity of a particular biological process (e.g., cell adhesion, P-selectin binding) in a cell. In some embodiments, the term refers to a reduction in P-selectin binding to a level that is statistically significantly lower than an initial level, which may, for example, be a baseline level of binding. In some embodiments, the “inhibition,” “inhibiting,” “inhibit,” or “inhibitor” refer to a reduction of the level of binding (e.g., P-selectin binding) to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of binding.

The term “binding affinity” refers to the strength of the binding interaction between a glycopeptide and a target (e.g., a protein, selectin, or P-selectin). Binding affinity can be expressed as the equilibrium dissociation constant, K_{d} (expressed in units of concentration, e.g., M, mM, μM, nM) associated with each binding interaction between the glycopeptide and a target (e.g., a protein, selectin, or P-selectin). The K_{d} value is directly related to the relationship of dissociation kinetics divided by the association kinetics. Binding affinities and K_{d} values are inversely related such that lower binding affinities correspond to higher K_{d} values, while higher binding affinities correspond to lower Ks values.

The term “half-life” refers to a biological half-life of a particular compound in vivo. Half-life (t_{1/2}) may be expressed as the time required for elimination of half of the dose administered to a subject from the blood and/or other tissues, that is, the time required for the maximum concentration to decrease to half maximum concentration. Half-life is typically used when the rate of removal is approximately exponential. In some embodiments, half-life may be measured by monitoring plasma concentration over time after intravenous administration of a single weight-based dose.

The term “IC₅₀” refers to the inhibitory concentration (e.g., M, mM, μM, nM) required to inhibit a biological or biochemical function by 50%. IC₅₀ is a quantitative measurement of the quantity of a particular inhibitor required to inhibit a biological process (e.g., cell adhesion, P-selecting binding) or process component (e.g., a protein, selectin, or P-selectin) by half.

As used herein, the term “similar” (e.g., “similar efficacy” “similar potency,” “similar binding affinity,” or “similar to”) refers to a degree of correspondence between two or more elements being compared. In some instances, elements can be considered to be similar based on determining whether the difference between the two elements is within a particular threshold or baseline value. Exemplary thresholds of deviation are typically within ±10%, or more typically within *5%, ±4%, *3%, ±2%, or ±1% of a given value or range of values being compared. In certain embodiments (e.g., binding affinity (K_{d})), exemplary thresholds of deviation are typically within 20-fold, 10-fold, 5-fold, or 2-fold. In some instances, “similar” may indicate there is no significant statistical difference between the quantities being compared.

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2%, or 1% of a given value or range of values.

Unless otherwise required by context, singular terms shall include pluralities, and plural terms shall include the singular.

### DETAILED DESCRIPTION OF CERTAIN EMBODIMENTS

Provided herein are glycopeptides (e.g., glycopeptides of SEQ ID NO: 1), and salts thereof, and pharmaceutical compositions thereof. Also provided herein are methods of treating and/or preventing a disease (e.g., cardiovascular disease, atherosclerosis, atherosclerotic lesions, thrombus formation, thromboembolism, stroke, myocardial infarction, allergy, lung disease, asthma, bronchitis, emphysema, and chronic obstructive pulmonary disease (COPD)) in a subject comprising administering a therapeutically effective amount of a compound or composition provided herein to the subject. The compound or composition may be administered as a monotherapy or in combination with another therapy, as described herein. Also described herein are methods of inhibiting P-selectin in a cell, tissue, or biological sample.

### Compounds

Provided herein are glycopeptide compounds of the formula

Y¹X¹Y²X²X³Y³X⁴X⁵X⁶Z¹X⁷W¹ (SEQ ID NO: 1),

or a salt thereof, wherein:
W¹ is threonine or serine substituted with a saccharide or polysaccharide,
X¹, X², X³, X⁴, X⁵, X⁶, and X⁷ are each individually and independently any amino acid,
Y¹, Y², and Y³ are each individually and independently tyrosine, phenylalanine, or phenylglycine, and wherein Y¹, Y², and Y³ are each independently unsubstituted or substituted with —SO₃H, —CH₂SO₃H, —CF₂SO₃H, —CO₂H, —CONH₂, —NHSO₂CH₃, —SO₂NH₂, or —CH₂PO₃H;
wherein at least one of Y¹, Y², and Y³ is substituted with —CH₂SO₃H;
Z¹ is proline or hydroxyproline; and
at least one amino acid residue is substituted with -L¹-PEG:
wherein L¹ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, or combinations thereof; and
PEG is linear or branched polyethylene glycol.

In certain embodiments, a salt of a glycopeptide provided herein is a pharmaceutically acceptable salt.

As defined herein, W¹ is threonine or serine substituted with a saccharide or polysaccharide. In certain embodiments, W¹ is threonine substituted with a saccharide or polysaccharide. In some embodiments, W¹ is serine substituted with a saccharide or polysaccharide. In certain embodiments, W¹ is threonine substituted with a saccharide. In some embodiments, W¹ is threonine substituted with a polysaccharide. In certain embodiments, W¹ is serine substituted with a saccharide. In some embodiments, W¹ is serine substituted with a polysaccharide.

As defined herein, Y¹, Y², and Y³ are each individually and independently tyrosine, phenylalanine, or phenylglycine, and wherein Y¹, Y², and Y³ are each independently unsubstituted or substituted with —SO₃H, —CH₂SO₃H, —CF₂SO₃H, —CO₂H, —CONH₂, —NHSO₂CH₃, —SO₂NH₂, or —CH₂PO₃H, wherein at least one of Y¹, Y², and Y³ is substituted with —CH₂SO₃H. In certain embodiments, Y¹, Y², and Y³ are each individually and independently tyrosine, phenylalanine, or phenylglycine, wherein Y¹, Y², and Y³ are each independently unsubstituted or substituted with —CH₂SO₃H.

In certain embodiments, at least one of Y¹, Y², and Y³ is substituted with —CH₂SO₃H. In some embodiments, Y¹ is substituted with —CH₂SO₃H. In certain embodiments, Y² is substituted with —CH₂SO₃H. In some embodiments, Y³ is substituted with —CH₂SO₃H. In certain embodiments, Y¹ and Y² are substituted with —CH₂SO₃H. In some embodiments, Y¹ and Y³ are substituted with —CH₂SO₃H. In certain embodiments, Y² and Y³ are substituted with —CH₂SO₃H.

In some embodiments. Y¹, Y², and Y³ are substituted with —CH₂SO₃H.

In certain embodiments, at least one of Y¹, Y², and Y³ is phenylalanine substituted with —CH₂SO₃H. In some embodiments, at least two of Y¹, Y², and Y³ are phenylalanine substituted with —CH₂SO₃H. In certain embodiments, Y¹, Y², and Y³ are phenylalanine substituted with —CH₂SO₃H.

As defined herein, X¹, X³, X⁴, and X⁷ are each individually and independently E. D, N, or Q. In certain embodiments, X¹, X³, X⁴, and X⁷ are each individually and independently E or D. In some embodiments. X¹ and X⁷ are E. In certain embodiments, X³ and X⁴ are D. In some embodiments, X¹ and X⁷ are E. and X³ and X⁴ are D.

As defined herein, X², X⁵, and X⁶ are each individually and independently L, F, V, A, or I. In certain embodiments, X², X⁵, and X⁶ are each individually and independently L or F. In some embodiments, X² and X⁶ are L. In certain embodiments, X⁵ is F. In some embodiments, X² and X⁶ are L, and X⁵ is F.

As defined herein, Z¹ is proline or hydroxyproline. In some embodiments, Z¹ is proline. In certain embodiments, Z¹ is hydroxyproline.

In certain embodiments, the glycopeptide of claim 1, or a salt thereof, comprises

(SEQ ID NO: 2)
Y¹LEY²LDY³DFLZ¹EW¹,
(SEQ ID NO: 3)
Y¹EY²LDY³DFLZ¹EW¹EP,
(SEQ ID NO: 4)
Y¹EY²LDY³DFLZ¹EW¹EPL,
(SEQ ID NO: 5)
EY¹EY²LDY³DFLZ¹EW¹,
(SEQ ID NO: 6)
EY¹EY¹LDY³DFLZEWIE,
(SEQ ID NO: 7)
EY¹EY²LDY³DFLZ¹EW¹EP,
(SEQ ID NO: 8)
EY¹EY²LDY³DFLZ¹EWIEPL,
(SEQ ID NO: 9)
KEY¹EY²LDY³DFLZ¹EW¹,
(SEQ ID NO: 10)
KEY¹EY²LDY³DFLZ¹EW¹E,
(SEQ ID NO: 11)
KEY¹EY²LDY³DFLZ¹EW¹EP,
or
(SEQ ID NO: 12)
KEY¹EY²LDY³DFLZ¹EW¹EPL.

In certain embodiments, the saccharide or polysaccharide comprises one or more sugars selected from the group consisting of: galactose, fucose, 2-(acetylamino)-2-deoxy-galactose, 2-(acetylamino)-2-deoxy-glucose, and 5-acetamido-3,5-dideoxy-glycero-galacto-2-nonulosonic acid.

In certain embodiments, the polysaccharide comprises 2-(acetylamino)-2-deoxy-galactose alpha 1 bonded to W¹, galactose beta 3 bonded to 2-(acetylamino)-2-deoxy-galactose, 2-(acetylamino)-2-deoxy-glucose beta 6 bonded to 2-(acetylamino)-2-deoxy-galactose, and fucose alpha 3 bonded to 2-(acetylamino)-2-deoxyglucose.

In certain embodiments, the polysaccharide comprises 2-(acetylamino)-2-deoxy-galactose alpha 1 bonded to W¹, galactose beta 3 bonded to 2-(acetylamino)-2-deoxy-galactose, 2-(acetylamino)-2-deoxy-glucose beta 6 bonded to 2-(acetylamino)-2-deoxy-galactose, fucose alpha 3 bonded to 2-(acetylamino)-2-deoxyglucose, and galactose beta 4 bonded to 2-(acetylamino)-2-deoxy-glucose.

In certain embodiments, the polysaccharide comprises 2-(acetylamino)-2-deoxy-galactose alpha 1 bonded to W¹, galactose beta 3 bonded to 2-(acetylamino)-2-deoxy-galactose, 2-(acetylamino)-2-deoxy-glucose beta 6 bonded to 2-(acetylamino)-2-deoxy-galactose, fucose alpha 3 bonded to 2-(acetylamino)-2-deoxyglucose, galactose beta 4 bonded to 2-(acetylamino)-2-deoxy-glucose, and 5-acetamido-3,5-dideoxy-glycero-galacto-2-nonulosonic acid alpha 3 bonded to galactose.

In certain embodiments, the polysaccharide comprises 2-(acetylamino)-2-deoxy-galactose alpha 1 bonded to W¹, galactose beta 3 bonded to 2-(acetylamino)-2-deoxy-galactose, 2-(acetylamino)-2-deoxy-glucose beta 6 bonded to 2-(acetylamino)-2-deoxy-galactose, galactose beta 4 bonded to 2-(acetylamino)-2-deoxy-glucose, 5-acetamido-3,5-dideoxy-glycero-galacto-2-nonulosonic acid alpha 3 bonded to galactose, and fucose alpha 3 bonded to 2-(acetylamino)-2-deoxy-glucose.

In certain embodiments, the polysaccharide comprises 2-(acetylamino)-2-deoxy-galactose alpha 1 bonded to W¹, a first galactose beta 3 bonded to 2-(acetylamino)-2-deoxy-galactose, 2-(acetylamino)-2-deoxy-glucose beta 6 bonded to 2-(acetylamino)-2-deoxy-galactose, a second galactose beta 4 bonded to 2-(acetylamino)-2-deoxy-glucose, 5-acetamido-3,5-dideoxy-glycero-galacto-2-nonulosonic acid alpha 3 bonded to the second galactose, fucose alpha 3 bonded to 2-(acetylamino)-2-deoxy-glucose, and 5-acetamido-3,5-dideoxy-glycero-galacto-2-nonulosonic acid alpha 3 bonded to the first galactose.

In certain embodiments, the polysaccharide comprises 2-(acetylamino)-2-deoxy-galactose alpha 1 bonded to W¹, a first galactose beta 3 bonded to 2-(acetylamino)-2-deoxy-galactose, 2-(acetylamino)-2-deoxy-glucose beta 6 bonded to 2-(acetylamino)-2-deoxy-galactose, fucose alpha 3 bonded to 2-(acetylamino)-2-deoxyglucose, and a second galactose beta 4 bonded to 2-(acetylamino)-2-deoxy-glucose.

In certain embodiments, the polysaccharide comprises 2-(acetylamino)-2-deoxy-galactose alpha 1 bonded to W¹, a first galactose beta 3 bonded to 2-(acetylamino)-2-deoxy-galactose, 2-(acetylamino)-2-deoxy-glucose beta 6 bonded to 2-(acetylamino)-2-deoxy-galactose, fucose alpha 3 bonded to 2-(acetylamino)-2-deoxyglucose, a second galactose beta 4 bonded to 2-(acetylamino)-2-deoxy-glucose, and 5-acetamido-3,5-dideoxy-glycero-galacto-2-nonulosonic acid alpha 3 bonded to the second galactose.

In certain embodiments, the polysaccharide is sialyl Lewis X or sialyl Lewis A. In some embodiments, the polysaccharide is sialyl Lewis X. In certain embodiments, the polysaccharide is sialyl Lewis A.

In certain embodiments, the glycopeptide, or a salt thereof, comprises the formula KEY¹EY²LDY³DFLZ¹EW¹EPL (SEQ ID NO: 12) wherein:
W¹ is threonine substituted with a polysaccharide,
Y¹, Y², and Y³ are phenylalanine 4-substituted with —CH₂SO₃H,
Z¹ is proline,
the polysaccharide comprises 2-(acetylamino)-2-deoxy-galactose alpha 1 bonded to W¹, a first galactose beta 3 bonded to 2-(acetylamino)-2-deoxy-galactose, 2-(acetylamino)-2-deoxy-glucose beta 6 bonded to 2-(acetylamino)-2-deoxy-galactose, fucose alpha 3 bonded to 2-(acetylamino)-2-deoxyglucose, a second galactose beta 4 bonded to 2-(acetylamino)-2-deoxy-glucose, and 5-acetamido-3,5-dideoxy-glycero-galacto-2-nonulosonic acid alpha 3 bonded to the first galactose; and
at least one amino acid residue is substituted with -L¹-PEG.

In certain embodiments, at least one instance of K, E, or D is substituted with -L¹-PEG. In some embodiments, exactly one instance of K, E, or D is substituted with -L¹-PEG. In certain embodiments, K is substituted with -L¹-PEG. In some embodiments, at least one instance of E is substituted with -L¹-PEG. In certain embodiments, exactly one instance of E is substituted with -L¹-PEG. In some embodiments, D is substituted with -L¹-PEG.

In certain embodiments, the PEG comprises

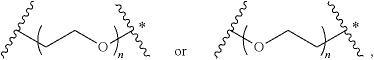

wherein “*” is attached to L¹. In some embodiments, the PEG comprises

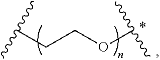

wherein “*” is
attached to L¹. In certain embodiments, the PEG comprises

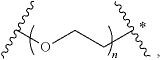

wherein “*” is attached to L¹.

In certain embodiments, the glycopeptide comprises a PEG that is linear or branched. In some embodiments, the glycopeptide comprises a PEG that is linear. In certain embodiments, the glycopeptide comprises a PEG that is branched. In some embodiments, the glycopeptide having a branched PEG comprises -L¹-(PEG)ₓ, wherein L¹ is substituted with x instances of PEG and x is an integer from 3 to 100. In some embodiments, the glycopeptide having a branched PEG comprises -L¹-(PEG)ₓ, wherein L¹ is substituted with x instances of PEG and x is an integer from 3 to 20.

In certain embodiments, n is an integer between 20 and 10,000, inclusive. In some embodiments, n is an integer between 20 and 5,000, inclusive. In certain embodiments, n is an integer between 20 and 3,500, inclusive. In some embodiments, n is an integer between 20 and 2,500, inclusive. In certain embodiments, n is an integer between 20 and 1,500, inclusive. In some embodiments, n is an integer between 100 and 3,500, inclusive. In certain embodiments, n is an integer between 100 and 2,500, inclusive. In some embodiments, n is an integer between 100 and 1,500, inclusive. In certain embodiments, n is an integer between 350 and 3,500, inclusive. In some embodiments, n is an integer between 350 and 2,500, inclusive. In certain embodiments, n is an integer between 350 and 1,500, inclusive. In some embodiments, n is an integer between 500 and 3,500, inclusive. In certain embodiments, n is an integer between 500 and 2,500, inclusive. In some embodiments, n is an integer between 500 and 1,500, inclusive.

In certain embodiments, the PEG comprises

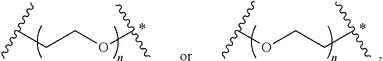

wherein “*” is attached to L¹ and the terminus not attached to L¹ terminates in an end group. In some embodiments, the PEG comprises

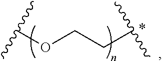

wherein “*” is attached to L¹ and the terminus not attached to L¹ terminates in an end group selected from in hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or an oxygen protecting group. In certain embodiments, the PEG comprises

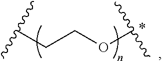

wherein “*” is attached to L¹ and the terminus not attached to L¹ terminates in an end group selected from hydroxy, substituted or unsubstituted alkoxy, substituted or unsubstituted alkeneoxy, substituted or unsubstituted alkynoxy, substituted or unsubstituted aryloxy, protected hydroxy, substituted or unsubstituted amine, thiol, thioether, or halogen. In some embodiments, the PEG comprises

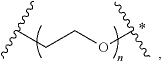

wherein “*” is attached to L¹ and the terminus not attached to L¹ terminates in an end group selected from hydroxy, protected hydroxy, or unsubstituted alkoxy. In certain embodiments, the PEG comprises

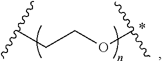

wherein “*” is attached to L¹ and the terminus not attached to L¹ terminates in an end group selected from —OH, —OCH₃, and —OCH₂CH₃. In some embodiments, the PEG comprises

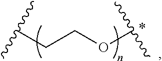

wherein “*” is attached to L¹ and the terminus not attached to L¹ terminates in an —OH end group. In certain embodiments, the PEG comprises

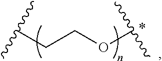

wherein “*” is attached to L¹ and the terminus not attached to L¹ terminates in an —OCH₃ end group. In some embodiments, the PEG comprises

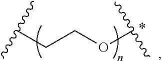

wherein “*” is attached to L¹ and the terminus not attached to L¹ terminates in an —OCH₂CH₃ end group.

As defined herein, L¹ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, or combinations thereof. In certain embodiments, L¹ comprises substituted or unsubstituted aliphatic. In some embodiments, L¹ comprises substituted or unsubstituted C₁₋₅₀ aliphatic. In certain embodiments, L¹ comprises substituted or unsubstituted C₁₋₁₀ aliphatic. In some embodiments, L¹ comprises substituted C₁₋₁₀ aliphatic. In certain embodiments, L¹ comprises unsubstituted C₁₋₁₀ aliphatic. In some embodiments, L¹ comprises substituted or unsubstituted C₁₋₁₀ alkylene. In certain embodiments, L¹ comprises unsubstituted C₁₋₁₀ alkylene.

In some embodiments, L¹ is unsubstituted C₁₋₁₀ alkylene. In certain embodiments, L¹ comprises substituted C₁₋₁₀ alkylene. In some embodiments, L¹ comprises C₁₋₁₀ alkylene substituted with a carbonyl. In certain embodiments, L¹ is C₁₋₁₀ alkylene substituted with a carbonyl. In some embodiments, L¹ comprises C₁₋₁₀ alkylene substituted with a hydroxy group. In certain embodiments, L¹ is C₁₋₁₀ alkylene substituted with a hydroxy group.

In certain embodiments, L¹ comprises substituted or unsubstituted heteroaliphatic. In some embodiments, L¹ comprises substituted or unsubstituted C₁₋₅₀ heteroaliphatic. In certain embodiments, L¹ comprises substituted or unsubstituted C₁₋₁₀ heteroaliphatic. In some embodiments, L¹ comprises substituted C₁₋₁₀ heteroaliphatic. In certain embodiments, L¹ comprises unsubstituted C₁₋₁₀ heteroaliphatic. In some embodiments, L¹ comprises substituted or unsubstituted C₁₋₁₀ heteroalkylene. In certain embodiments, L¹ comprises unsubstituted C₁₋₁₀ heteroalkylene. In some embodiments, L¹ is unsubstituted C₁₋₁₀ heteroalkylene. In certain embodiments, L¹ is unsubstituted C₁₋₁₀ heteroalkylene comprising one or more oxygen, nitrogen, or sulfur atoms. In some embodiments, L¹ is unsubstituted C₁₋₁₀ heteroalkylene comprising one or more oxygen atoms. In certain embodiments, L¹ is unsubstituted C₁₋₁₀ heteroalkylene comprising one or more nitrogen atoms. In some embodiments, L¹ is unsubstituted C₁₋₁₀ heteroalkylene comprising one or more sulfur atoms. In certain embodiments. L¹ comprises substituted C₁₋₁₀ heteroalkylene. In some embodiments, L¹ is substituted C₁₋₁₀ heteroalkylene. In certain embodiments. L¹ is substituted C₁₋₁₀ heteroalkylene comprising one or more oxygen, nitrogen, or sulfur atoms. In some embodiments, L¹ is C₁₋₁₀ heteroalkylene comprising one or more oxygen, nitrogen, or sulfur atoms substituted with one or more carbonyl groups. In some embodiments, L¹ is C₁₋₁₀ heteroalkylene comprising one or more oxygen atoms and substituted with one or more carbonyl groups. In certain embodiments, L¹ is C₁₋₁₀ heteroalkylene comprising one or more nitrogen atoms and substituted with one or more carbonyl groups. In some embodiments, L¹ is C₁₋₁₀ heteroalkylene comprising one or more sulfur atoms substituted with one or more carbonyl groups.

In certain embodiments, L¹ comprises substituted or unsubstituted carbocyclylene. In some embodiments, L¹ comprises unsubstituted carbocyclylene. In certain embodiments, L¹ comprises substituted carbocyclylene. In some embodiments, L¹ comprises C₃0.6 substituted carbocyclylene. In certain embodiments, L¹ comprises C₅₋₆ carbocyclylene.

In certain embodiments, L¹ comprises substituted or unsubstituted heterocyclylene. In some embodiments, L¹ comprises substituted heterocyclylene. In certain embodiments, L¹ comprises unsubstituted heterocyclylene. In some embodiments. L¹ comprises C₅₋₆ substituted heterocyclylene.

In certain embodiments, L¹ comprises substituted or unsubstituted arylene. In some embodiments, L¹ comprises substituted arylene. In certain embodiments, L¹ comprises unsubstituted arylene. In some embodiments, L¹ comprises unsubstituted C₆₋₁₄ arylene. In certain embodiments, L¹ comprises substituted C₆₋₁₄ arylene.

In certain embodiments, L¹ comprises substituted or unsubstituted heteroarylene. In some embodiments, L¹ comprises substituted heteroarylene. In certain embodiments, L¹ comprises unsubstituted heteroarylene. In some embodiments, L¹ comprises a substituted 5-6 membered heteroarylene. In certain embodiments. L¹ comprises an unsubstituted 5-6 membered heteroarylene. In some embodiments, L¹ comprises a substituted 1,3,5-triazine.

In certain embodiments, L¹ has the structure:

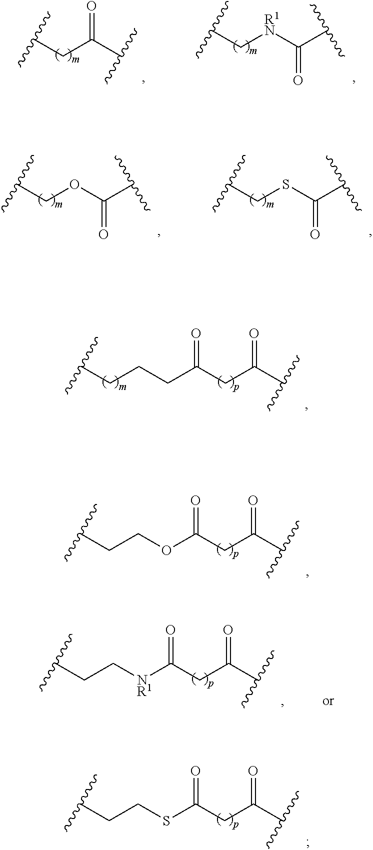

wherein
m is 0 to 20;
p is 1 to 20: and
R¹ is hydrogen or alkyl.

In certain embodiments, L¹ has the structure:

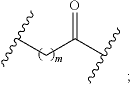

wherein m is 0-20. In some embodiments, L¹ has the structure:

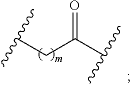

wherein m is 0-10. In certain embodiments, L¹ has the structure:

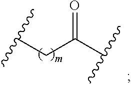

wherein m is 1. In some embodiments, L¹ has the structure:

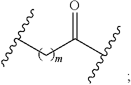

wherein m is 2. In certain embodiments, L¹ has the structure:

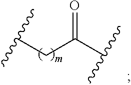

wherein m is 3. In some embodiments, L¹ has the structure:

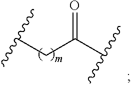

wherein m is 4.

In certain embodiments, L¹ has the structure:

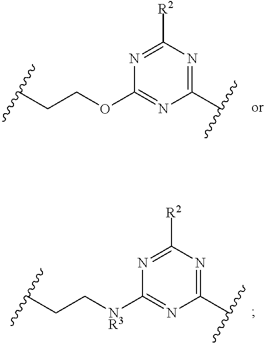

wherein
R¹ is hydrogen or alkyl; and
R² is —H, a halogen, —OR³, —N(R³)₂, —SR³, or —C(R³)₃, wherein R³ is —H, substituted or unsubstituted aliphatic, substituted or unsubstituted heteroaliphatic, substituted or unsubstituted aryl, substituted or unsubstituted heteroaryl, substituted or unsubstituted carbocyclic, or substituted or unsubstituted heterocyclic, or combinations thereof.

In certain embodiments, L¹ has the structure:

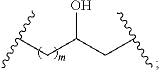

wherein m is 0 to 20.

In certain embodiments, L¹ has the structure:

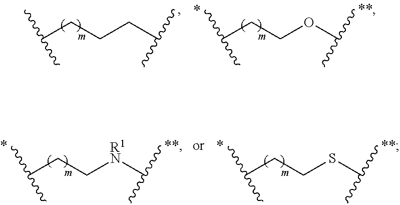

wherein
m is 0 to 20;
R¹ is hydrogen or alkyl;
“*” is attached to the polyethylene glycol moiety; and
“**” is attached to the glycopeptide.

In certain embodiments, L¹ has the structure:

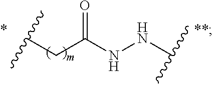

wherein
m is 0 to 20;
“*” is attached to the polyethylene glycol moiety; and
“**” is attached to the glycopeptide.

As defined herein, m is 0 to 20. In some embodiments, m is 0 to 10. In certain embodiments, m is 0 to 5. In some embodiments m is 10. In certain embodiments m is 9. In some embodiments m is 8. In certain embodiments m is 7. In some embodiments m is 6. In certain embodiments m is 5. In some embodiments m is 4. In certain embodiments m is 3. In some embodiments m is 2. In certain embodiments m is 1. In some embodiments, m is 0.

As defined herein, p is 1 to 20. In some embodiments, p is 1 to 10. In certain embodiments, p is 1 to 5. In some embodiments p is 10. In certain embodiments p is 9. In some embodiments p is 8. In certain embodiments p is 7. In some embodiments p is 6. In certain embodiments p is 5. In some embodiments p is 4. In certain embodiments p is 3. In some embodiments p is 2. In certain embodiments p is 1.

As defined herein, R¹ is hydrogen or alkyl. In some embodiments, R¹ is hydrogen. In certain embodiments, R¹ is alkyl. In some embodiments, R¹ is C₁₋₆ alkyl. In certain embodiments, R¹ is C₁₋₆ unsubstituted alkyl. In some embodiments. R¹ is n-butyl, n-propyl, ethyl, methyl, or hydrogen. In certain embodiments, R¹ is n-butyl, n-propyl, ethyl, or methyl. In some embodiments, R¹ is n-butyl. In certain embodiments, R¹ is n-propyl. In some embodiments, R¹ is ethyl. In certain embodiments, R¹ is methyl.

As defined herein, R² is R² is —H, a halogen, —OR₃, —N(R³)₂, —SR³, or —C(R³)₃. In certain embodiments, R² is —H. In some embodiments. R² is a halogen. In certain embodiments. R² is —Cl. In certain embodiments, R² is —OR³. In some embodiments. R² is —N(R₃)₂. In certain embodiments, R² is —SR³. In some embodiments, R² is —C(R³)₃.

As defined herein, R³ is —H, substituted or unsubstituted aliphatic, substituted or unsubstituted heteroaliphatic, substituted or unsubstituted aryl, substituted or unsubstituted heteroaryl, substituted or unsubstituted carbocyclic, or substituted or unsubstituted heterocyclic, or combinations thereof. In certain embodiments, R³ is —H. In some embodiments, R³ comprises substituted or unsubstituted aliphatic. In some embodiments, R³ comprises substituted or unsubstituted C₁₋₁₀ aliphatic. In certain embodiments, R³ comprises substituted or unsubstituted heteroaliphatic. In some embodiments, R³ comprises substituted or unsubstituted C₁₋₁₀ heteroaliphatic. In certain embodiments, R³ comprises substituted or unsubstituted aryl. In some embodiments, R³ comprises substituted or unsubstituted C₆₋₁₄ aryl. In certain embodiments, R³ comprises substituted or unsubstituted heteroaryl. In some embodiments, R³ comprises substituted or unsubstituted 5-6 membered heteroaryl. In certain embodiments, R³ comprises substituted or unsubstituted carbocyclic. In some embodiments, R³ comprises substituted or unsubstituted C₃₋₇ carbocyclic. In certain embodiments, R³ comprises substituted or unsubstituted heterocyclic. In some embodiments, R³ comprises substituted or unsubstituted C₃₋₇ heterocyclic.

In certain embodiments, the glycopeptide comprises the formula

KEY¹EY²LDY³DFLZ¹EW¹EPL (SEQ ID NO: 12), wherein:
W¹ is threonine substituted with a polysaccharide,
Y¹, Y², and Y³ are phenylalanine 4-substituted with —CH₂SO₃H,
Z¹ is proline,
the polysaccharide comprises 2-(acetylamino)-2-deoxy-galactose alpha 1 bonded to W¹, a first galactose beta 3 bonded to 2-(acetylamino)-2-deoxy-galactose, 2-(acetylamino)-2-deoxy-glucose beta 6 bonded to 2-(acetylamino)-2-deoxy-galactose, fucose alpha 3 bonded to 2-(acetylamino)-2-deoxyglucose, a second galactose beta 4 bonded to 2-(acetylamino)-2-deoxy-glucose, and 5-acetamido-3,5-dideoxy-glycero-galacto-2-nonulosonic acid alpha 3 bonded to the first galactose,
K is substituted with -L¹-PEG, wherein
the PEG is linear and terminates in —OCH₃; and
L¹ has the structure

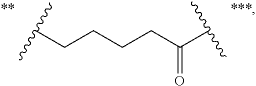 [SMILES: *CCCCC(*)=O]

wherein “**” is attached to PEG, and
“***” is attached to K.

In certain embodiments, the glycopeptide comprises the formula

KEY¹EY²LDY³DFLZ¹EW¹EPL (SEQ ID NO: 12), wherein:
W¹ is threonine substituted with a polysaccharide,
Y¹, Y², and Y³ are phenylalanine 4-substituted with —CH₂SO₃H,
Z¹ is proline,
the polysaccharide comprises 2-(acetylamino)-2-deoxy-galactose alpha 1 bonded to W¹, a first galactose beta 3 bonded to 2-(acetylamino)-2-deoxy-galactose, 2-(acetylamino)-2-deoxy-glucose beta 6 bonded to 2-(acetylamino)-2-deoxy-galactose, fucose alpha 3 bonded to 2-(acetylamino)-2-deoxyglucose, a second galactose beta 4 bonded to 2-(acetylamino)-2-deoxy-glucose, and 5-acetamido-3,5-dideoxy-glycero-galacto-2-nonulosonic acid alpha 3 bonded to the first galactose,
K is substituted with -L¹-PEG, wherein
the PEG is branched and terminates in —OCH₃; and
L¹ has the structure

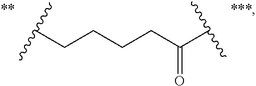 [SMILES: *CCCCC(*)=O]

wherein “**” is attached to PEG, and
“***” is attached to K.

In certain embodiments, the glycopeptide, or salt thereof, is characterized as having similar efficacy to that of the corresponding non-PEGylated glycopeptide. In some embodiments, the similar efficacy comprises similar potency. In certain embodiments, the similar efficacy comprises binding affinity (K_{d}) for P-selectin and half-life (t_{1/2}) that are similar to the binding affinity (K_{d}) for P-selectin and half-life (t_{1/2}) of the corresponding non-PEGylated glycopeptide.

In some embodiments, the similar efficacy comprises lower binding affinity (K_{d}) for P-selectin and increased half-life (t_{1/2}) relative to the binding affinity (K_{d}) for P-selectin and half-life (t_{1/2}), respectively, of the corresponding non-PEGylated glycopeptide. In certain embodiments, the similar efficacy comprises a binding affinity (K_{d}) for P-selectin that is similar to the binding affinity (K_{d}) for P-selectin of the corresponding non-PEGylated glycopeptide. In some embodiments, the similar efficacy comprises increased half-life (t½) relative to the half-life (t_{1/2}) of the corresponding non-PEGylated glycopeptide.

In certain embodiments, the glycopeptide, or salt thereof, is characterized as having a binding affinity (K_{d}) for P-selectin similar to the binding affinity (K_{d}) for P-selectin of the corresponding non-PEGylated glycopeptide. In some embodiments, the glycopeptide, or salt thereof, is characterized as having a binding affinity (K_{d}) for P-selectin up to 20-fold greater than that of the corresponding non-PEGylated glycopeptide. In certain embodiments, the glycopeptide, or salt thereof, is characterized as having a binding affinity (K_{d}) for P-selectin up to 10-fold greater than that of the corresponding non-PEGylated glycopeptide. In some embodiments, the glycopeptide, or salt thereof, is characterized as having a binding affinity (K_{d}) for P-selectin up to 5-fold greater than that of the corresponding non-PEGylated glycopeptide. In certain embodiments, the glycopeptide, or salt thereof, is characterized as having a binding affinity (K_{d}) for P-selectin up to 2-fold greater than that of the corresponding non-PEGylated glycopeptide.

In certain embodiments, the glycopeptide, or salt thereof, binds P-selectin with a binding affinity (K_{d}) of up to 5 nM. In some embodiments, the glycopeptide, or salt thereof, binds P-selectin with a binding affinity (K_{d}) of up to 10 nM. In certain embodiments, the glycopeptide, or salt thereof, binds P-selectin with a binding affinity (K_{d}) of up to 15 nM. In some embodiments, the glycopeptide, or salt thereof, binds P-selectin with a binding affinity (K_{d}) of up to 20 nM. In certain embodiments, the glycopeptide, or salt thereof, binds P-selectin with a binding affinity (K_{d}) of up to 25 nM. In some embodiments, the glycopeptide, or salt thereof, binds P-selectin with a binding affinity (K_{d}) of up to 30 nM. In certain embodiments, the glycopeptide, or salt thereof, binds P-selectin with a binding affinity (K_{d}) of up to 35 nM. In some embodiments, the glycopeptide, or salt thereof, binds P-selectin with a binding affinity (K_{d}) of up to 40 nM. In certain embodiments, the glycopeptide, or salt thereof, binds P-selectin with a binding affinity (K_{d}) of up to 50 nM. In some embodiments, the glycopeptide, or salt thereof, binds P-selectin with a binding affinity (K_{d}) of up to 60 nM. In certain embodiments, the glycopeptide, or salt thereof, binds P-selectin with a binding affinity (K_{d}) of up to 70 nM. In some embodiments, the glycopeptide, or salt thereof, binds P-selectin with a binding affinity (K_{d}) of up to 80 nM. In certain embodiments, the glycopeptide, or salt thereof, binds P-selectin with a binding affinity (K_{d}) of up to 100 nM.

In certain embodiments, the glycopeptide, or salt thereof, is characterized as having an increased half-life (t_{1/2}) relative to the half-life (t_{1/2}) of the corresponding non-PEGylated glycopeptide. In some embodiments, the glycopeptide, or salt thereof, has a half-life (t_{1/2}) of at least 5 minutes. In certain embodiments, the glycopeptide, or salt thereof, has a half-life (t_{1/2}) of at least 10 minutes. In some embodiments, the glycopeptide, or salt thereof, has a half-life (t_{1/2}) of at least 30 minutes. In certain embodiments, the glycopeptide, or salt thereof, has a half-life (t_{1/2}) of at least 1 hour. In some embodiments, the glycopeptide, or salt thereof, has a half-life (t_{1/2}) of at least 2 hours. In certain embodiments, the glycopeptide, or salt thereof, has a half-life (t_{1/2}) of at least 3 hours. In some embodiments, the glycopeptide, or salt thereof, has a half-life (t_{1/2}) of at least 4 hours. In certain embodiments, the glycopeptide, or salt thereof, has a half-life (t_{1/2}) of at least 5 hours. In certain embodiments, the glycopeptide, or salt thereof, has a half-life (t_{1/2}) of at least 6 hours. In some embodiments, the glycopeptide, or salt thereof, has a half-life (t_{1/2}) of at least 7 hours. In certain embodiments, the glycopeptide, or salt thereof, has a half-life (t½) of at least 8 hours. In some embodiments, the glycopeptide, or salt thereof, has a half-life (t_{1/2}) of at least 9 hours. In certain embodiments, the glycopeptide, or salt thereof, has a half-life (t½) of at least 10 hours. In some embodiments, the glycopeptide, or salt thereof, has a half-life (t_{1/2}) of at least 11 hours. In certain embodiments, the glycopeptide, or salt thereof, has a half-life (t½) of at least 12 hours. In some embodiments, the glycopeptide, or salt thereof, has a half-life (t_{1/2}) of at least 24 hours. In certain embodiments, the glycopeptide, or salt thereof, has a half-life (t_{1/2}) of at least 48 hours. In some embodiments, the glycopeptide, or salt thereof, has a half-life (t_{1/2}) of at least 72 hours. In certain embodiments, the glycopeptide, or salt thereof, has a half-life (t½) of at least a week. In some embodiments, the glycopeptide, or salt thereof, has a half-life (t_{1/2}) of at least two weeks. In certain embodiments, the glycopeptide, or salt thereof, has a half-life (t_{1/2}) of at least three weeks.

In certain embodiments, the glycopeptide, or salt thereof, is characterized as having an IC₅₀ for P-selectin that is similar to the IC50 for P-selectin of the corresponding non-PEGylated glycopeptide. In some embodiments, the glycopeptide, or salt thereof, has an IC₅₀ for P-selectin of 1-100 μM. In certain embodiments, the glycopeptide, or salt thereof, has an IC₅₀ for P-selectin of 1-50 μM. In some embodiments, the glycopeptide, or salt thereof, has an IC₅₀ for P-selectin of 1-30 μM. In certain embodiments, the glycopeptide, or salt thereof, has an IC₅₀ for P-selectin of 1-20 μM. In some embodiments, the glycopeptide, or salt thereof, has an IC₅₀ for P-selectin of 1-10 μM. In certain embodiments, the glycopeptide, or salt thereof, has an IC₅₀ for P-selectin of 1-5 μM. In some embodiments, the glycopeptide, or salt thereof, has an IC₅₀ for P-selectin of approximately 1 μM. In certain embodiments, the glycopeptide, or salt thereof, has an IC₅₀ for P-selectin of approximately 2 μM. In some embodiments, the glycopeptide, or salt thereof, has an IC₅₀ for P-selectin of approximately 3 μM. In certain embodiments, the glycopeptide, or salt thereof, has an IC₅₀ for P-selectin of approximately 4 μM. In some embodiments, the glycopeptide, or salt thereof, has an IC₅₀ for P-selectin of approximately 5 μM. In certain embodiments, the glycopeptide, or salt thereof, has an IC₅₀ for P-selectin of approximately 6 μM. In some embodiments, the glycopeptide, or salt thereof, has an IC₅₀ for P-selectin of approximately 7 μM. In certain embodiments, the glycopeptide, or salt thereof, has an IC₅₀ for P-selectin of approximately 8 μM. In some embodiments, the glycopeptide, or salt thereof, has an IC₅₀ for P-selectin of approximately 9 μM. In certain embodiments, the glycopeptide, or salt thereof, has an IC₅₀ for P-selectin of approximately 10 μM. In some embodiments, the glycopeptide, or salt thereof, has an IC₅₀ for P-selectin of approximately 15 μM. In certain embodiments, the glycopeptide, or salt thereof, has an IC₅₀ for P-selectin of approximately 20 μM. In some embodiments, the glycopeptide, or salt thereof, has an IC₅₀ for P-selectin of approximately 25 μM. In certain embodiments, the glycopeptide, or salt thereof, has an IC₅₀ for P-selectin of approximately 30 μM.

### Pharmaceutical Compositions, Kits, and Administration

The present disclosure provides pharmaceutical compositions comprising a glycopeptide of (SEQ ID NO: 1), or a salt thereof, and optionally a pharmaceutically acceptable excipient. In certain embodiments, the pharmaceutical composition described herein comprises a (SEQ ID NO: 1), or a salt thereof, and a pharmaceutically acceptable excipient.

In certain embodiments, the glycopeptide described herein, or a salt thereof, is provided in an effective amount in the pharmaceutical composition. In some embodiments, the effective amount is a therapeutically or prophylactically effective amount. In certain embodiments, the effect amount is an amount effective for treating a disease (e.g., cardiovascular disease, atherosclerosis, atherosclerotic lesions, thrombus formation, thromboembolism, stroke, myocardial infarction, allergy, lung disease, asthma, bronchitis, emphysema, and chronic obstructive pulmonary disease (COPD)) in a subject in need thereof. In certain embodiments, the effective amount is an amount effective for preventing a disease (e.g., cardiovascular disease, atherosclerosis, atherosclerotic lesions, thrombus formation, thromboembolism, stroke, myocardial infarction, allergy, lung disease, asthma, bronchitis, emphysema, and chronic obstructive pulmonary disease (COPD)) in a subject in need thereof. In some embodiments, the effective amount is an amount effective for reducing the risk of developing a disease (e.g., cardiovascular disease, atherosclerosis, atherosclerotic lesions, thrombus formation, thromboembolism, stroke, myocardial infarction, allergy, lung disease, asthma, bronchitis, emphysema, and chronic obstructive pulmonary disease (COPD)) in a subject in need thereof.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit. In certain embodiments, a pharmaceutical composition described herein could be prepared according to the known method such as a method described in the general rules for preparations of the Japanese Pharmacopoeia, 16th edition, the United States Pharmacopoeia, and the European Pharmacopoeia, 9th edition. A pharmaceutical composition of the disclosure could be administered to patients appropriately depending on the dosage form.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition. In certain embodiments, the pharmaceutically acceptable excipient is a saccharide or polysaccharide.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate. D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid. (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin. (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically, contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein.

In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., potency and/or efficacy) in treating a disease in a subject in need thereof, in preventing a disease in a subject in need thereof, in reducing the risk to develop a disease in a subject in need thereof, and/or in inhibiting P-selectin in a subject or cell, improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

The compound or composition can be administered concurrently with, prior to, or subsequent to one or more additional pharmaceutical agents, which may be useful as, e.g., combination therapies. Pharmaceutical agents include therapeutically active agents.

Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., cardiovascular disease, atherosclerosis, atherosclerotic lesions, thrombus formation, thromboembolism, stroke, myocardial infarction, allergy, lung disease, asthma, bronchitis, emphysema, and chronic obstructive pulmonary disease (COPD)). Each additional pharmaceutical agent may be administered at a dose and/or on a time schedule determined for that pharmaceutical agent. The additional pharmaceutical agents may also be administered together with each other and/or with the compound or composition described herein in a single dose or composition or administered separately in different doses or compositions. The particular combination to employ in a regimen will take into account compatibility of the compound described herein with the additional pharmaceutical agent(s) and/or the desired therapeutic and/or prophylactic effect to be achieved.

In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins.

Anti-inflammatory agents include, but are not limited to, salicylates, aspirin, diflunisal, salsalate, propionic acid derivatives, ibuprofen, dexibuprofen, naproxen, fenoprofen, ketoprofen, dexketoprofen, flurbiprofen, oxaprozin, loxoprofen, acetic acid derivatives, indomethacin, tolmetin, sulindac, etodolac, ketorolac, diclofenac, nabumetone, enolic acid (oxicam) derivatives, piroxicam, meloxicam, tenoxicam, droxicam, lornoxicam, isoxicam, fenamic acid derivatives (fenamates), mefenamic acid, meclofenamic acid, flufenamic acid, tolfenamic acid, selective COX-2 inhibitors (voxibs), celecoxib, rofecoxib, valdecoxib, parecoxib, lumiracoxib, etoricoxib, firocoxib, sulphonanilides, nimesulide, licofelone, and combinations thereof.

Cardiovascular agents include, but are not limited to, a statin, atorvastatin, cerivastatin, Fluvastatin, lovastatin, mevastatin, pitavastatin, pravastatin, rosuvastatin, simvastatin, ezetimibe, amlodipine, niacin, aspirin, omega-3 fatty acid, or combinations thereof.

Anti-cancer agents encompass biotherapeutic anti-cancer agents as well as chemotherapeutic agents.

Exemplary biotherapeutic anti-cancer agents include, but are not limited to, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g. HERCEPTIN (trastuzumab), T-DM1, AVASTIN (bevacizumab), ERBITUX (cetuximab), VECTIBIX (panitumumab), RITUXAN (rituximab). BEXXAR (tositumomab)).

Exemplary chemotherapeutic agents include, but are not limited to, anti-estrogens (e.g. tamoxifen, raloxifene, and megestrol), LHRH agonists (e.g. goserelin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

Additional pharmaceutical agents include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells.

In certain embodiments, the pharmaceutical composition is in the form of a pill, tablet, capsule, or gel. In some embodiments, the pharmaceutical composition is in the form of a pill. In certain embodiments, the pharmaceutical composition is in the form of a tablet. In some embodiments, the pharmaceutical composition is in the form of a capsule. In certain embodiments, the pharmaceutical composition is in the form of a gel. In certain embodiments, the pharmaceutical composition is in the form of an aqueous saline buffer. In some embodiments, the pharmaceutical composition is in the form of an aqueous saline buffer, wherein the pharmaceutically acceptable excipient is a saccharide or polysaccharide. In certain embodiments, the pharmaceutical composition is in the form of an aqueous saline buffer, wherein the pharmaceutically acceptable excipient is a saccharide. In some embodiments, the pharmaceutical composition is in the form of an aqueous saline buffer, wherein the pharmaceutically acceptable excipient is a polysaccharide.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., cardiovascular disease, atherosclerosis, atherosclerotic lesions, thrombus formation, thromboembolism, stroke, myocardial infarction, allergy, lung disease, asthma, bronchitis, emphysema, and chronic obstructive pulmonary disease (COPD)) in a subject in need thereof. In certain embodiments, the kits are useful for preventing a disease (e.g., cardiovascular disease, atherosclerosis, atherosclerotic lesions, thrombus formation, thromboembolism, stroke, myocardial infarction, allergy, lung disease, asthma, bronchitis, emphysema, and chronic obstructive pulmonary disease (COPD)) in a subject in need thereof. In certain embodiments, the kits are useful for reducing the risk of developing a disease (e.g., cardiovascular disease, atherosclerosis, atherosclerotic lesions, thrombus formation, thromboembolism, stroke, myocardial infarction, allergy, lung disease, asthma, bronchitis, emphysema, and chronic obstructive pulmonary disease (COPD)) in a subject in need thereof. In certain embodiments, the kits are useful for inhibiting P-selectin in a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., cardiovascular disease, atherosclerosis, atherosclerotic lesions, thrombus formation, thromboembolism, stroke, myocardial infarction, allergy, lung disease, asthma, bronchitis, emphysema, and chronic obstructive pulmonary disease (COPD)) in a subject in need thereof. In certain embodiments, the kits and instructions provide for preventing a disease (e.g., cardiovascular disease, atherosclerosis, atherosclerotic lesions, thrombus formation, thromboembolism, stroke, myocardial infarction, allergy, lung disease, asthma, bronchitis, emphysema, and chronic obstructive pulmonary disease (COPD)) in a subject in need thereof. In certain embodiments, the kits and instructions provide for reducing the risk of developing a disease (e.g., cardiovascular disease, atherosclerosis, atherosclerotic lesions, thrombus formation, thromboembolism, stroke, myocardial infarction, allergy, lung disease, asthma, bronchitis, emphysema, and chronic obstructive pulmonary disease (COPD)) in a subject in need thereof. In certain embodiments, the kits and instructions provide for inhibiting P-selectin in a subject or cell. A kit described herein may include one or more additional pharmaceutical agents described herein as a separate composition.

### Methods of Treatment and Uses

Provided herein are methods of using the glycopeptides provided herein (e.g., glycopeptides of SEQ ID NO: 1), and salts thereof, and pharmaceutical compositions thereof.

Provided herein are methods of treating and/or preventing a disease (e.g., cardiovascular disease, atherosclerosis, atherosclerotic lesions, thrombus formation, thromboembolism, stroke, myocardial infarction, allergy, lung disease, asthma, bronchitis, emphysema, and chronic obstructive pulmonary disease (COPD)) in a subject in need thereof, the methods comprising administering to the subject a glycopeptide of SEQ ID No: 1, or a salt thereof, or a pharmaceutical composition thereof. In certain embodiments, the disease is cardiovascular disease, atherosclerosis, atherosclerotic lesions, thrombus formation, thromboembolism, stroke, myocardial infarction, or cancer. In some embodiments, the disease is an allergy or a lung disease. In certain embodiments, the disease is asthma, bronchitis, emphysema, and chronic obstructive pulmonary disease (COPD).

Provided herein are uses of the glycopeptides of SEQ ID No: 1, salts thereof, and pharmaceutical compositions thereof, for the preparation of a medicament for treating and/or preventing a disease (e.g., cardiovascular disease, atherosclerosis, atherosclerotic lesions, thrombus formation, thromboembolism, stroke, myocardial infarction, allergy, lung disease, asthma, bronchitis, emphysema, and chronic obstructive pulmonary disease (COPD)). In certain embodiments, the disease is cardiovascular disease, atherosclerosis, atherosclerotic lesions, thrombus formation, thromboembolism, stroke, myocardial infarction, or cancer. In some embodiments, the disease is an allergy or a lung disease. In certain embodiments, the disease is asthma, bronchitis, emphysema, and chronic obstructive pulmonary disease (COPD).

Also provided herein are glycopeptides of SEQ ID No: 1, salts thereof, and pharmaceutical compositions thereof for use in treating and/or preventing a disease(e.g., cardiovascular disease, atherosclerosis, atherosclerotic lesions, thrombus formation, thromboembolism, stroke, myocardial infarction, allergy, lung disease, asthma, bronchitis, emphysema, and chronic obstructive pulmonary disease (COPD)) in a subject. In certain embodiments, the disease is cardiovascular disease, atherosclerosis, atherosclerotic lesions, thrombus formation, thromboembolism, stroke, myocardial infarction, or cancer. In some embodiments, the disease is an allergy or a lung disease. In certain embodiments, the disease is asthma, bronchitis, emphysema, and chronic obstructive pulmonary disease (COPD).

In certain embodiments, the disease is cardiovascular disease, atherosclerosis, atherosclerotic lesions, thrombus formation, thromboembolism, stroke, myocardial infarction, or cancer. In some embodiments, the disease is cardiovascular disease. In certain embodiments, the disease is atherosclerosis or atherosclerotic lesions. In some embodiments, the disease is thrombus formation or thromboembolism. In certain embodiments, the disease is atherosclerosis.

In some embodiments, the disease is atherosclerotic lesions. In certain embodiments, the disease is thrombus formation. In some embodiments, the disease is thromboembolism. In certain embodiments, the disease is a stroke. In some embodiments, the disease is myocardial infarction. In certain embodiments, the disease is cancer.

In certain embodiments, the cancer is venous ulcers, angiogenic disorders of the skin, a hematological malignancy, a leukemia, lymphoma, acute lymphoblastic leukemia (ALL), acute myelogenous leukemia (AML), chronic lymphocytic leukemia (CLL), small lymphocytic lymphoma (SLL), chronic myelogenous leukemia, acute monocytic leukemia (AMOL), Hodgkin's lymphomas, non-Hodgkin's lymphomas, Burkitt lymphoma, B-cell lymphoma, multiple myelomacervical, ovarian cancer, colon cancer, breast cancer, gastric cancer, lung cancer, melanoma, skin cancer, pancreatic cancer, prostate cancer, head cancer, neck cancer, or renal cancer.

In certain embodiments, the disease is an allergy or lung disease. In some embodiments, the disease is an allergy. In certain embodiments, the disease is a lung disease. In some embodiments, the disease is asthma, bronchitis, emphysema, and chronic obstructive pulmonary disease (COPD). In certain embodiments, the disease is asthma. In some embodiments, the disease is bronchitis. In certain embodiments, the disease is emphysema. In some embodiments, the disease is chronic obstructive pulmonary disease (COPD).

In certain embodiments, the subject is at risk of, exhibiting symptoms of, or diagnosed with atherosclerosis, atherosclerotic lesions, thrombus formation, thromboembolism, stroke, myocardial infarction, or cancer. In some embodiments, the subject is at risk of, exhibiting symptoms of, or diagnosed with asthma, bronchitis, emphysema, and chronic obstructive pulmonary disease (COPD).

In certain embodiments, the effective amount of the glycopeptides, or salts thereof, and pharmaceutical compositions thereof is administered orally, parenterally, intramuscularly, subcutaneously, intravenously, topically, or transdermally. In some embodiments, the effective amount of the glycopeptide, or a salt thereof, or a pharmaceutical composition thereof is administered orally. In certain embodiments, the effective amount of the glycopeptide, or a salt thereof, or a pharmaceutical composition thereof is administered parenterally. In some embodiments the effective amount of the glycopeptide, or a salt thereof, or a pharmaceutical composition thereof is administered intramuscularly. In certain embodiments, the effective amount of the glycopeptide, or a salt thereof, or a pharmaceutical composition thereof is administered subcutaneously. In some embodiments, the effective amount of the glycopeptide, or a salt thereof, or a pharmaceutical composition thereof is administered intravenously. In certain embodiments, the effective amount of the glycopeptide, or a salt thereof, or a pharmaceutical composition thereof is administered topically. In some embodiments, the effective amount of the glycopeptide, or a salt thereof, or a pharmaceutical composition thereof is administered transdermally.

In certain embodiments, the glycopeptide, or salt thereof, is characterized as having similar efficacy to that of the corresponding non-PEGylated glycopeptide. In some embodiments, the similar efficacy comprises similar potency. In certain embodiments, the similar efficacy comprises binding affinity (K_{d}) for P-selectin and half-life (t½) that are similar to the binding affinity (K_{d}) for P-selectin and half-life (t_{1/2}) of the corresponding non-PEGylated glycopeptide. In some embodiments, the similar efficacy comprises lower binding affinity (K_{d}) for P-selectin and increased half-life (t_{1/2}) relative to the binding affinity (K_{d}) for P-selectin and half-life (t_{1/2}) , respectively, of the corresponding non-PEGylated glycopeptide. In certain embodiments, the similar efficacy comprises a binding affinity (K_{d}) for P-selectin that is similar to the binding affinity (K_{d}) for P-selectin of the corresponding non-PEGylated glycopeptide. In some embodiments, the similar efficacy comprises increased half-life (t_{1/2}) relative to the half-life (t½) of the corresponding non-PEGylated glycopeptide.

In certain embodiments, the glycopeptide, or salt thereof, is characterized as having a binding affinity (K_{d}) for P-selectin similar to the binding affinity (K_{d}) for P-selectin of the corresponding non-PEGylated glycopeptide. In some embodiments, the glycopeptide, or salt thereof, is characterized as having a binding affinity (K_{d}) for P-selectin up to 20-fold greater than that of the corresponding non-PEGylated glycopeptide. In certain embodiments, the glycopeptide, or salt thereof, is characterized as having a binding affinity (K_{d}) for P-selectin up to 10-fold greater than that of the corresponding non-PEGylated glycopeptide. In some embodiments, the glycopeptide, or salt thereof, is characterized as having a binding affinity (K_{d}) for P-selectin up to 5-fold greater than that of the corresponding non-PEGylated glycopeptide. In certain embodiments, the glycopeptide, or salt thereof, is characterized as having a binding affinity (K_{d}) for P-selectin up to 2-fold greater than that of the corresponding non-PEGylated glycopeptide.

In certain embodiments, the glycopeptide, or salt thereof, binds P-selectin with a binding affinity (K_{d}) of up to 5 nM. In some embodiments, the glycopeptide, or salt thereof, binds P-selectin with a binding affinity (K_{d}) of up to 10 nM. In certain embodiments, the glycopeptide, or salt thereof, binds P-selectin with a binding affinity (K_{d}) of up to 15 nM. In some embodiments, the glycopeptide, or salt thereof, binds P-selectin with a binding affinity (K_{d}) of up to 20 nM. In certain embodiments, the glycopeptide, or salt thereof, binds P-selectin with a binding affinity (K_{d}) of up to 25 nM. In some embodiments, the glycopeptide, or salt thereof, binds P-selectin with a binding affinity (K_{d}) of up to 30 nM. In certain embodiments, the glycopeptide, or salt thereof, binds P-selectin with a binding affinity (K_{d}) of up to 35 nM. In some embodiments, the glycopeptide, or salt thereof, binds P-selectin with a binding affinity (K_{d}) of up to 40 nM. In certain embodiments, the glycopeptide, or salt thereof, binds P-selectin with a binding affinity (K_{d}) of up to 50 nM. In some embodiments, the glycopeptide, or salt thereof, binds P-selectin with a binding affinity (K_{d}) of up to 60 nM. In certain embodiments, the glycopeptide, or salt thereof, binds P-selectin with a binding affinity (K_{d}) of up to 70 nM. In some embodiments, the glycopeptide, or salt thereof, binds P-selectin with a binding affinity (K_{d}) of up to 80 nM. In certain embodiments, the glycopeptide, or salt thereof, binds P-selectin with a binding affinity (K_{d}) of up to 100 nM.

In certain embodiments, the glycopeptide, or salt thereof, is characterized as having an increased half-life (t_{1/2}) relative to the half-life (t_{1/2}) of the corresponding non-PEGylated glycopeptide. In some embodiments, the glycopeptide, or salt thereof, has a half-life (t_{1/2}) of at least 5 minutes. In certain embodiments, the glycopeptide, or salt thereof, has a half-life (t_{1/2}) of at least 10 minutes. In some embodiments, the glycopeptide, or salt thereof, has a half-life (t_{1/2}) of at least 30 minutes. In certain embodiments, the glycopeptide, or salt thereof, has a half-life (t_{1/2}) of at least 1 hour. In some embodiments, the glycopeptide, or salt thereof, has a half-life (t_{1/2}) of at least 2 hours. In certain embodiments, the glycopeptide, or salt thereof, has a half-life (t½) of at least 3 hours. In some embodiments, the glycopeptide, or salt thereof, has a half-life (t_{1/2}) of at least 4 hours. In certain embodiments, the glycopeptide, or salt thereof, has a half-life (t_{1/2}) of at least 5 hours. In certain embodiments, the glycopeptide, or salt thereof, has a half-life (t½) of at least 6 hours. In some embodiments, the glycopeptide, or salt thereof, has a half-life (t½) of at least 7 hours. In certain embodiments, the glycopeptide, or salt thereof, has a half-life (t_{1/2}) of at least 8 hours. In some embodiments, the glycopeptide, or salt thereof, has a half-life (t½) of at least 9 hours. In certain embodiments, the glycopeptide, or salt thereof, has a half-life (t½) of at least 10 hours. In some embodiments, the glycopeptide, or salt thereof, has a half-life (t_{1/2}) of at least 11 hours. In certain embodiments, the glycopeptide, or salt thereof, has a half-life (t½) of at least 12 hours. In some embodiments, the glycopeptide, or salt thereof, has a half-life (t_{1/2}) of at least 24 hours. In certain embodiments, the glycopeptide, or salt thereof, has a half-life (t½) of at least 48 hours. In some embodiments, the glycopeptide, or salt thereof, has a half-life (t_{1/2}) of at least 72 hours. In certain embodiments, the glycopeptide, or salt thereof, has a half-life (t_{1/2}) of at least a week. In some embodiments, the glycopeptide, or salt thereof, has a half-life (t_{1/2}) of at least two weeks. In certain embodiments, the glycopeptide, or salt thereof, has a half-life (t_{1/2}) of at least three weeks.

In certain embodiments, the glycopeptide, or salt thereof, has a similar IC₅₀ for a protein as the corresponding non-PEGylated glycopeptide. In some embodiments, the protein is P-selectin. In certain embodiments, the glycopeptide, or salt thereof, has an IC₅₀ for P-selectin that is similar to the IC₅₀ for P-selectin of the corresponding non-PEGylated glycopeptide. In some embodiments, the glycopeptide, or salt thereof, has an IC₅₀ for P-selectin of 1-100 μM. In certain embodiments, the glycopeptide, or salt thereof, has an IC₅₀ for P-selectin of 1-50 μM. In some embodiments, the glycopeptide, or salt thereof, has an IC₅₀ for P-selectin of 1-30 μM. In certain embodiments, the glycopeptide, or salt thereof, has an IC₅₀ for P-selectin of 1-20 μM. In some embodiments, the glycopeptide, or salt thereof, has an IC₅₀ for P-selectin of 1-10 μM. In certain embodiments, the glycopeptide, or salt thereof, has an IC₅₀ for P-selectin of 1-5 μM. In some embodiments, the glycopeptide, or salt thereof, has an IC₅₀ for P-selectin of approximately 1 μM.

In certain embodiments, the glycopeptide, or salt thereof, has an IC₅₀ for P-selectin of approximately 2 μM. In some embodiments, the glycopeptide, or salt thereof, has an IC₅₀ for P-selectin of approximately 3 μM. In certain embodiments, the glycopeptide, or salt thereof, has an IC₅₀ for P-selectin of approximately 4 μM. In some embodiments, the glycopeptide, or salt thereof, has an IC₅₀ for P-selectin of approximately 5 μM. In certain embodiments, the glycopeptide, or salt thereof, has an IC₅₀ for P-selectin of approximately 6 μM. In some embodiments, the glycopeptide, or salt thereof, has an IC₅₀ for P-selectin of approximately 7 μM. In certain embodiments, the glycopeptide, or salt thereof, has an IC₅₀ for P-selectin of approximately 8 μM. In some embodiments, the glycopeptide, or salt thereof, has an IC₅₀ for P-selectin of approximately 9 μM. In certain embodiments, the glycopeptide, or salt thereof, has an IC₅₀ for P-selectin of approximately 10 μM. In some embodiments, the glycopeptide, or salt thereof, has an IC₅₀ for P-selectin of approximately 15 μM. In certain embodiments, the glycopeptide, or salt thereof, has an IC₅₀ for P-selectin of approximately 20 p M. In some embodiments, the glycopeptide, or salt thereof, has an IC₅₀ for P-selectin of approximately 25 μM. In certain embodiments, the glycopeptide, or salt thereof, has an IC₅₀ for P-selectin of approximately 30 p M.

### Additional Methods and Uses

The present disclosure also provides methods of inhibiting P-selectin comprising contacting a cell, tissue, or biological sample with an effective amount of a glycopeptide provided herein, or a salt thereof, or a pharmaceutical composition thereof.

Provided herein are methods of inhibiting P-selectin comprising contacting a cell, tissue, or biological sample with a glycopeptide of SEQ ID No: 1, or a salt thereof, or a pharmaceutical composition thereof. In certain embodiments, the glycopeptide, or a salt thereof, binds P-selectin.

In some embodiments, the glycopeptide, or a salt thereof, binds P-selectin with similar affinity to that of the corresponding non-PEGylated glycopeptide.

In some embodiments, any of the glycopeptides or compositions described herein are contacted with a cell in vivo, e.g., in an organism. In some embodiments, any of the glycopeptides or compositions described herein are contacted with a cell in vitro, e.g., in cell culture. In some embodiments, any of the glycopeptides or compositions described herein are contacted with a cell ex vivo, meaning the cell is removed from an organism prior to the contacting. As will be evident to one of skill in the art, the term cell may be used to refer to a single cell as well as a population of cells. In some embodiments, the populations or cells are contacted with any of the compounds described herein to regenerate or differentiate one or more cells in the population of cells. In some embodiments, the populations or cells are contacted with any of the glycopeptides described herein for use in personalized medicine, for example for diagnostic and/or therapeutic purposes.

In general, any cells known in the art may be used in the methods and uses described herein. In certain embodiments, the cell is of a cell line. In some embodiments, the cell is obtained from an organism, such as a subject. In certain embodiments, the cell is a leukocyte. In some embodiments, the cell is a monocyte. In certain embodiments, the cell is a neutrophil. In some embodiments, the cell is a human leukocyte. In certain embodiments, the cell is a human neutrophil. In some embodiments, the cell is a human monocyte. In certain embodiments, the cell is a mouse leukocyte. In some embodiments, the cell is a mouse neutrophil. In certain embodiments, the cell is a mouse monocyte. In some embodiments, the cell is a tumor cell. In certain embodiments, the cell is a tumor cell expressing ligands for P-selectin.

In some embodiments, the methods further comprise measuring or assessing the level of one or more properties of the cell. In some embodiments, the level of one or more properties of the cell is assessed following contacting the cell with any of the glycopeptides or compositions described herein. In some embodiments, the level of one or more properties following contacting the cell with any of the glycopeptides or compositions described herein is compared to the level of one or more properties in a reference sample or prior to contacting the cell with the glycopeptides or composition. In some embodiments, contacting the cell with any of the glycopeptides or compositions described herein increases one or more properties of the cell. In certain embodiments, contacting the cell with any of the glycopeptides or compositions described herein decreases one or more properties of the cell. In some embodiments, the methods described herein may be used to determine whether a cell is susceptible to treatment with the glycopeptides or compositions described herein. In some embodiments, if the level of one or more properties is increased following contacting the cell with any of the glycopeptides or compositions described herein, the cell is determined to be susceptible to treatment with the glycopeptides or composition. In some embodiments, if the level of one or more properties is increased following contacting the cell with any of the glycopeptides or compositions described herein, the compound or composition is determined to be a candidate for a disease or disorder associated with the cell.

In certain embodiments, the glycopeptide, or salt thereof, is characterized as having similar efficacy to that of the corresponding non-PEGylated glycopeptide. In some embodiments, the similar efficacy comprises similar potency. In certain embodiments, the similar efficacy comprises binding affinity (K_{d}) for P-selectin and half-life (t_{1/2}) that are similar to the binding affinity (K_{d}) for P-selectin and half-life (t_{1/2}) of the corresponding non-PEGylated glycopeptide.

In some embodiments, the similar efficacy comprises lower binding affinity (K_{d}) for P-selectin and increased half-life (t_{1/2}) relative to the binding affinity (K_{d}) for P-selectin and half-life (t_{1/2}), respectively, of the corresponding non-PEGylated glycopeptide. In certain embodiments, the similar efficacy comprises a binding affinity (K_{d}) for P-selectin that is similar to the binding affinity (K_{d}) for P-selectin of the corresponding non-PEGylated glycopeptide. In some embodiments, the similar efficacy comprises increased half-life (ta,) relative to the half-life (t½) of the corresponding non-PEGylated glycopeptide.

In certain embodiments, the glycopeptide, or salt thereof, is characterized as having a binding affinity (K_{d}) for P-selectin similar to the binding affinity (K_{d}) for P-selectin of the corresponding non-PEGylated glycopeptide. In some embodiments, the glycopeptide, or salt thereof, is characterized as having a binding affinity (K_{d}) for P-selectin up to 20-fold greater than that of the corresponding non-PEGylated glycopeptide. In certain embodiments, the glycopeptide, or salt thereof, is characterized as having a binding affinity (K_{d}) for P-selectin up to 10-fold greater than that of the corresponding non-PEGylated glycopeptide. In some embodiments, the glycopeptide, or salt thereof, is characterized as having a binding affinity (K_{d}) for P-selectin up to 5-fold greater than that of the corresponding non-PEGylated glycopeptide. In certain embodiments, the glycopeptide, or salt thereof, is characterized as having a binding affinity (K_{d}) for P-selectin up to 2-fold greater than that of the corresponding non-PEGylated glycopeptide.

In certain embodiments, the glycopeptide, or salt thereof, binds P-selectin with a binding affinity (K_{d}) of up to 5 nM. In some embodiments, the glycopeptide, or salt thereof, binds P-selectin with a binding affinity (K_{d}) of up to 10 nM. In certain embodiments, the glycopeptide, or salt thereof, binds P-selectin with a binding affinity (K_{d}) of up to 15 nM. In some embodiments, the glycopeptide, or salt thereof, binds P-selectin with a binding affinity (K_{d}) of up to 20 nM. In certain embodiments, the glycopeptide, or salt thereof, binds P-selectin with a binding affinity (K_{d}) of up to 25 nM. In some embodiments, the glycopeptide, or salt thereof, binds P-selectin with a binding affinity (K_{d}) of up to 30 nM. In certain embodiments, the glycopeptide, or salt thereof, binds P-selectin with a binding affinity (K_{d}) of up to 35 nM. In some embodiments, the glycopeptide, or salt thereof, binds P-selectin with a binding affinity (K_{d}) of up to 40 nM. In certain embodiments, the glycopeptide, or salt thereof, binds P-selectin with a binding affinity (K_{d}) of up to 50 nM. In some embodiments, the glycopeptide, or salt thereof, binds P-selectin with a binding affinity (K_{d}) of up to 60 nM. In certain embodiments, the glycopeptide, or salt thereof, binds P-selectin with a binding affinity (K_{d}) of up to 70 nM. In some embodiments, the glycopeptide, or salt thereof, binds P-selectin with a binding affinity (K_{d}) of up to 80 nM. In certain embodiments, the glycopeptide, or salt thereof, binds P-selectin with a binding affinity (K_{d}) of up to 100 nM.

In certain embodiments, the glycopeptide, or salt thereof, is characterized as having an increased half-life (t-ₕ) relative to the half-life (t_{1/2}) of the corresponding non-PEGylated glycopeptide. In some embodiments, the glycopeptide, or salt thereof, has a half-life (t_{1/2}) of at least 5 minutes. In certain embodiments, the glycopeptide, or salt thereof, has a half-life (t_{1/2}) of at least 10 minutes. In some embodiments, the glycopeptide, or salt thereof, has a half-life (t_{1/2}) of at least 30 minutes. In certain embodiments, the glycopeptide, or salt thereof, has a half-life (t_{1/2}) of at least 1 hour. In some embodiments, the glycopeptide, or salt thereof, has a half-life (t_{1/2}) of at least 2 hours. In certain embodiments, the glycopeptide, or salt thereof, has a half-life (t_{1/2}) of at least 3 hours. In some embodiments, the glycopeptide, or salt thereof, has a half-life (t_{1/2}) of at least 4 hours. In certain embodiments, the glycopeptide, or salt thereof, has a half-life (t_{1/2}) of at least 5 hours. In certain embodiments, the glycopeptide, or salt thereof, has a half-life (t_{1/2}) of at least 6 hours. In some embodiments, the glycopeptide, or salt thereof, has a half-life (t_{1/2}) of at least 7 hours. In certain embodiments, the glycopeptide, or salt thereof, has a half-life (t_{1/2}) of at least 8 hours. In some embodiments, the glycopeptide, or salt thereof, has a half-life (t_{1/2}) of at least 9 hours. In certain embodiments, the glycopeptide, or salt thereof, has a half-life (t_{1/2}) of at least 10 hours. In some embodiments, the glycopeptide, or salt thereof, has a half-life (t_{1/2}) of at least 11 hours. In certain embodiments, the glycopeptide, or salt thereof, has a half-life (t_{1/2}) of at least 12 hours. In some embodiments, the glycopeptide, or salt thereof, has a half-life (t_{1/2}) of at least 24 hours. In certain embodiments, the glycopeptide, or salt thereof, has a half-life (t_{1/2}) of at least 48 hours. In some embodiments, the glycopeptide, or salt thereof, has a half-life (t_{1/2}) of at least 72 hours. In certain embodiments, the glycopeptide, or salt thereof, has a half-life (t_{1/2}) of at least a week. In some embodiments, the glycopeptide, or salt thereof, has a half-life (t_{1/2}) of at least two weeks. In certain embodiments, the glycopeptide, or salt thereof, has a half-life (t_{1/2}) of at least three weeks.

In certain embodiments, the effective amount of the glycopeptide, or salt thereof, or the pharmaceutical composition thereof, produces greater than 50% inhibition. In some embodiments, the effective amount of the glycopeptide, or salt thereof, or the pharmaceutical composition thereof, produces greater than 60% inhibition. In certain embodiments, the effective amount of the glycopeptide, or salt thereof, or the pharmaceutical composition thereof, produces greater than 70% inhibition. In some embodiments, the effective amount of the glycopeptide, or salt thereof, or the pharmaceutical composition thereof, produces greater than 75% inhibition. In certain embodiments, the effective amount of the glycopeptide, or salt thereof, or the pharmaceutical composition thereof, produces greater than 80% inhibition. In some embodiments, the effective amount of the glycopeptide, or salt thereof, or the pharmaceutical composition thereof, produces greater than 85% inhibition. In certain embodiments, the effective amount of the glycopeptide, or salt thereof, or the pharmaceutical composition thereof, produces greater than 90% inhibition. In some embodiments, the effective amount of the glycopeptide, or salt thereof, or the pharmaceutical composition thereof, produces greater than 95% inhibition.

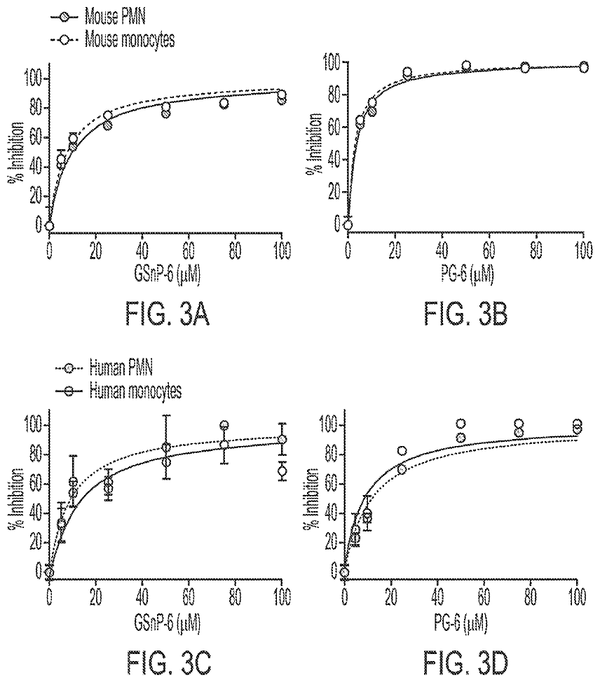

In certain embodiments, percent inhibition of P-selectin is characterized by flow cytometry (see FIGS. 3A-D). In some embodiments, percent binding inhibition of P-selectin chimera to neutrophils is characterized by flow cytometry (see FIGS. 3A-D). In certain embodiments, percent binding inhibition of P-selectin chimera to monocytes is characterized by flow cytometry (see FIGS. 3A-D).

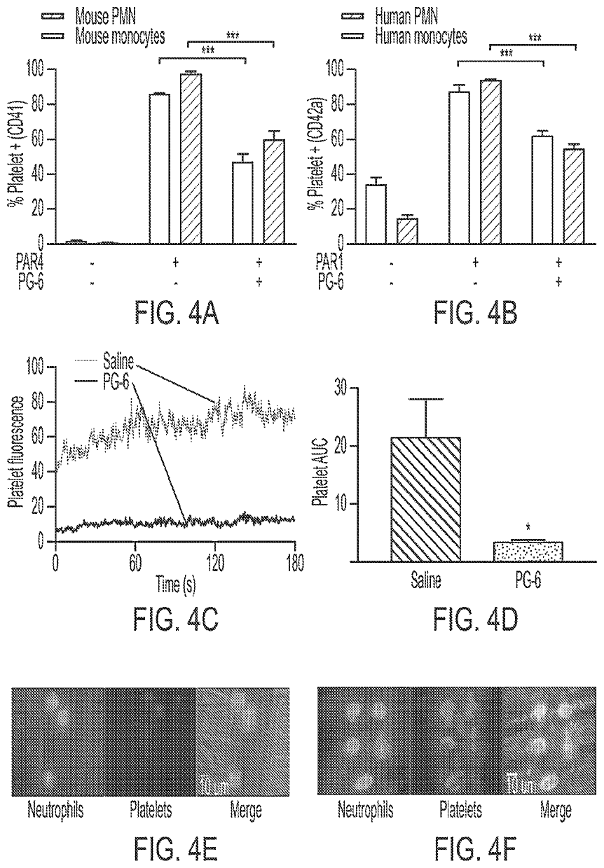

In certain embodiments, the methods inhibit platelet-leukocyte aggregation (see FIGS. 4A-F). In some embodiments, the methods inhibit platelet-leukocyte aggregation in vivo. In certain embodiments, the methods inhibit platelet-leukocyte aggregation in vitro. In some embodiments, the methods inhibit platelet accumulation to neutrophils. In certain embodiments, the methods inhibit platelet accumulation to monocytes. In some embodiments, platelet-leukocyte aggregation in vitro is characterized by flow cytometry (see FIGS. 4A-B). In certain embodiments, platelet-leukocyte aggregation in vitro is characterized by flow cytometry to quantify platelet-positive cells (see FIGS. 4A-B). In some embodiments, platelet-leukocyte aggregation in vivo is characterized by intravital microscopy (see FIGS. 4C-F). In certain embodiments, TNF-α-induced venular inflammation in vivo is characterized by intravital microscopy (see FIGS. 4C-F).

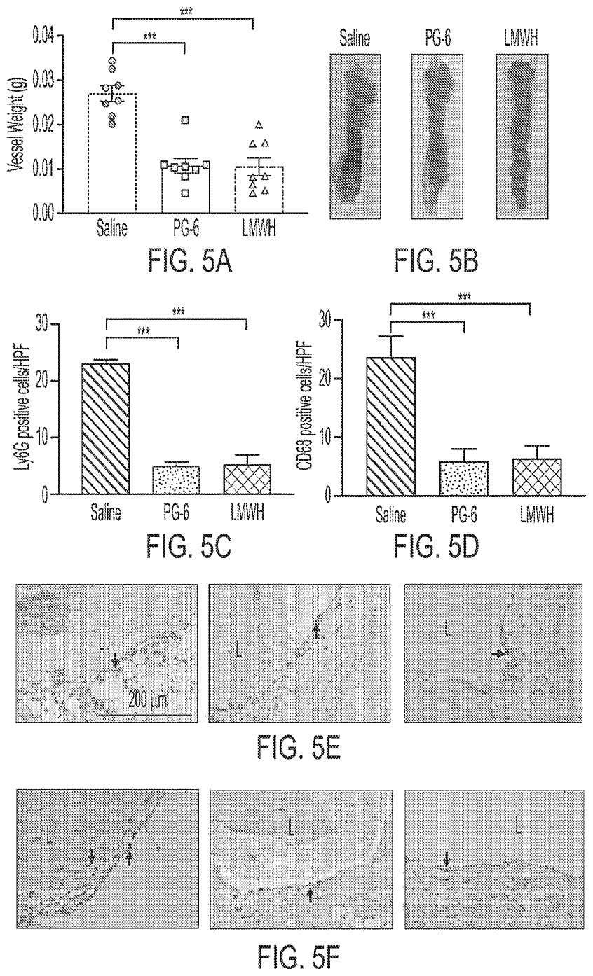

In certain embodiments, the methods reduce venous thrombus formation. In some embodiments, the methods reduce thrombus weight (see FIGS. 5A-B).

In certain embodiments, the methods reduce vein wall inflammation. In some embodiments, vein wall inflammation is characterized by neutrophil infiltration within the vein wall (see FIG. 5C and FIG. 5E). In certain embodiments, vein wall inflammation is characterized by macrophage infiltration within the vein wall (see FIG. 5D and FIG. 5F).

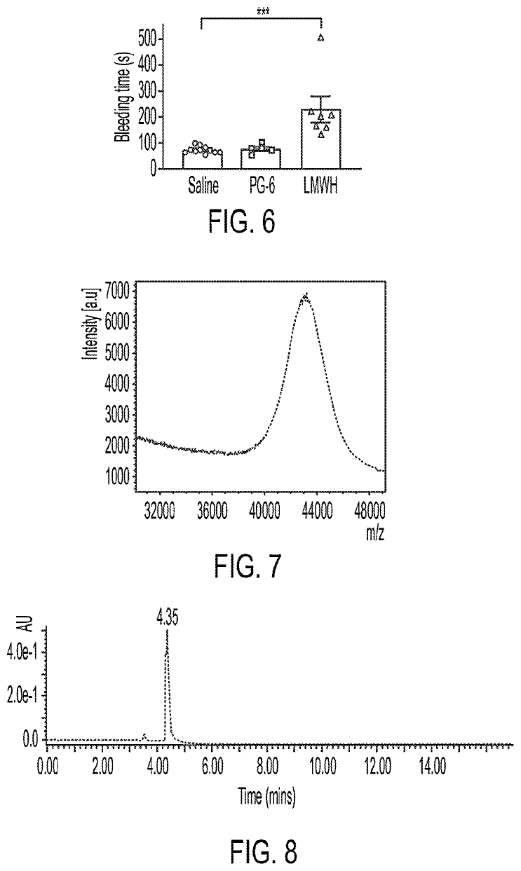

In certain embodiments, the methods do not affect hemostasis. In some embodiments, hemostasis is assessed using a tail vein bleeding assay (see FIG. 6). In certain embodiments, the methods do not significantly increase bleeding time (see FIG. 6).

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

Events mediated by the P-selectin/PSGL-1 pathway play a critical role in the initiation and propagation of venous thrombosis by facilitating the accumulation of leukocytes and platelets within the growing thrombus. Activated platelets and endothelium express P-selectin, which binds PSGL-1 expressed on the surface of all leukocytes. We developed a pegylated glycomimetic of the N-terminus of PSGL-1, PEG40-GSnP-6 (PG-6), is a highly potent P-selectin inhibitor with a favorable pharmacokinetic profile for clinical translation. PG-6 inhibits human and mouse platelet-monocyte and platelet-neutrophil aggregation in vitro and blocks microcirculatory platelet-leukocyte interactions in vivo. Administration of PG-6 reduces thrombus formation in a non-occlusive model of deep vein thrombosis with a commensurate reduction in leukocyte accumulation, but without disruption of hemostasis. PG-6 potently inhibits the P-selectin/PSGL-1 pathway and represents a promising drug candidate for the prevention or treatment of venous thrombosis without increased bleeding risk.

P-selectin is expressed on the surface of activated platelets and endothelium and binds its cognate ligand, P-selectin glycoprotein ligand-1 (PSGL-1), expressed on leukocytes, which supports leukocyte rolling and platelet-leukocyte aggregation. ¹⁹⁻²⁴ Transgenic mouse studies have demonstrated that P-selectin mediates leukocyte recruitment, tissue factor release, and fibrin deposition during the course of venous thrombosis.^{11,12,25,26} Significantly, in a variety of animal models, inhibitors of the P-selectin/PSGL-1 pathway, including monoclonal antibodies (mAbs),²⁷⁻²⁹ recombinant PSGL-1 (rPSGL-Ig),³⁰⁻³² small molecule antagonists,³³⁻³⁶ and oligonucleotide aptamers³⁷ have attenuated the VTE phenotype by reducing thrombus accumulation, accelerating clot resolution, and decreasing the local inflammatory response and late vein wall fibrosis.

While supporting the potential clinical significance of this pathway, these therapies face a number of barriers to widespread clinical use for VTE. All therapeutic antibodies are susceptible to an antidrug immune response that may promote the loss of drug efficacy, particularly among patients requiring repeated dosing and long-term therapy.³⁸ Evidence of anti-drug antibody production to both chimeric and humanized mAbs underscores the continued limitations to reducing antibody immunogenicity.³⁹ Recombinant protein production possesses its own inherent challenges, including imprecise post-translation modifications in non-human cell lines with a failure to preserve structural fidelity under sensitive culture conditions.⁴⁰ As an example, translational studies with rPSGL-Ig were only possible following the discovery that transfection of Chinese hamster ovarian cell lines with core-2β1,6-GlcNAc transferase was required to preserve high affinity bonding.⁴¹⁻⁴³ Moreover, protein modifications can induce hypersensitivity, alter pharmacokinetics, and induce immunogenicity^{44,45} As such, the production of recombinant proteins, especially therapeutic glycoproteins, requires precise control of an inherently complex and sensitive biologic process, which is especially difficult to achieve on a commercial scale.⁴⁶

While synthetic small molecule inhibitors for P-selectin remain an attractive target, developing high affinity synthetic antagonists continues to pose a significant challenge.⁴⁷ First generation P-selectin inhibitors were designed to mimic the tetrasaccharide siayl LewisX (sLeX) moiety of PSGL-1, but failed to account for the crucial contributions of multiple clustered tyrosine sulfates on the N-terminus of PSGL-1.^{36,48-53} High affinity binding of P-selectin to PSGL-1 requires stereospecific interactions with both clustered tyrosine sulfates and the sLeX-containing hexasaccharide epitope.⁵³⁻⁵⁵ To date, synthetic P-selectin antagonists exhibit relatively low micromolar affinity for P-selectin (GMI-1070 IC50 423 μM PSI-697 IC50150 M: PSI-421 IC50 225 μM)^{48,49} compared to nanomolar affinity observed for native P-selectin/PSGL-1 interactions (K_{d} 70-300 nM).^{56,57} As a consequence, the lack of clinical efficacy of these compounds has been attributed, at least in part, to their significantly diminished binding affinity. For example, despite demonstrated ex vivo efficacy, PSI-697 failed to inhibit platelet-monocyte aggregation in a phase I trial, whereas gram-scale dosing was required for GMI-1070 in clinical studies of sickle cell disease.⁵⁸⁻⁵⁹ Notably, crizanilizumab, the only approved P-selectin inhibitor, as an antibody, possesses a K_{d} in the low nanomolar range.⁶⁰

There is a need to develop P-selectin inhibitors that exhibit both high affinity and specificity. A rationally designed novel glycosulfopeptide (GSP) mimic of N-terminal PSGL-1, GSnP-6, was recently reported as the first synthetic high affinity (K_{d} 22 nM) and specific P-selectin antagonist for human therapy. GSnP-6 reduces leukocyte rolling and platelet-leukocyte aggregation in vitro and in vivo.⁶¹ In this report, the efficacy of a pegylated GSnP-6 (PG-6) is demonstrated in a preclinical model of non-occlusive venous thrombosis and confirm that this lead drug candidate does not disrupt hemostasis.

Results

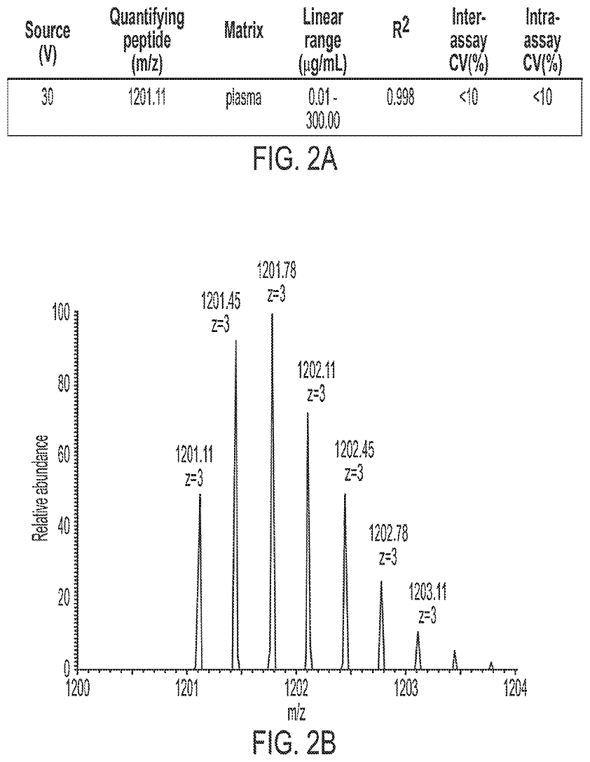

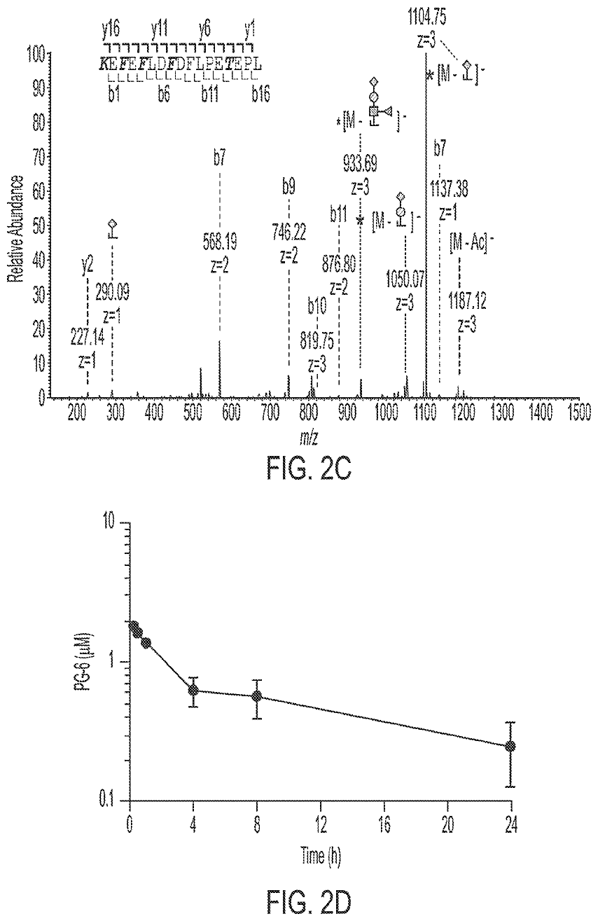

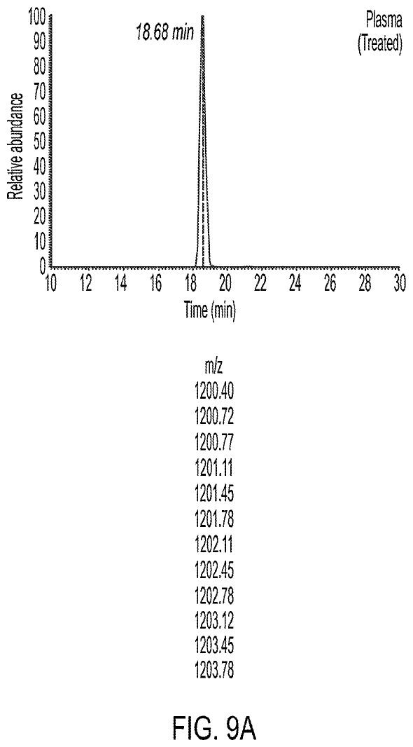

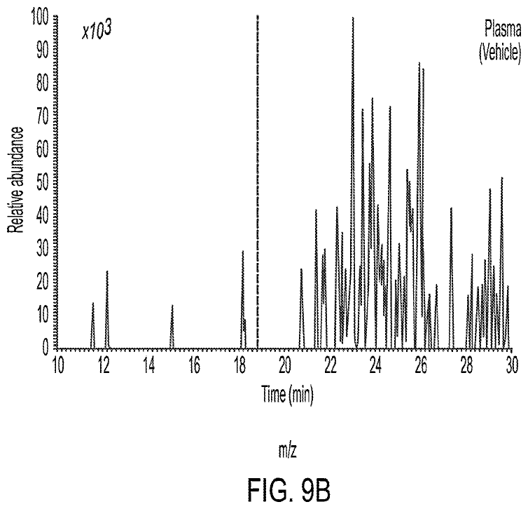

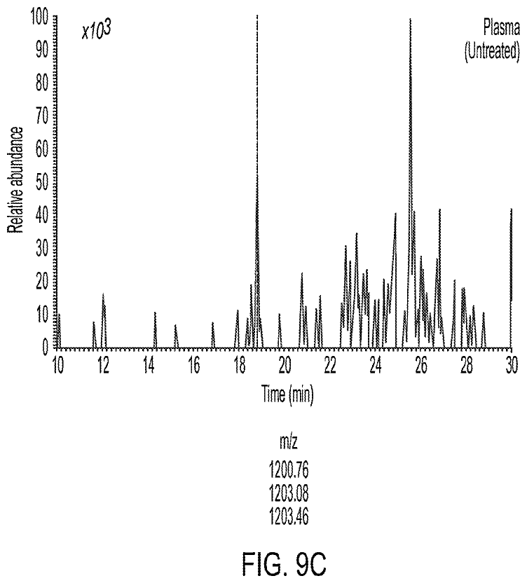

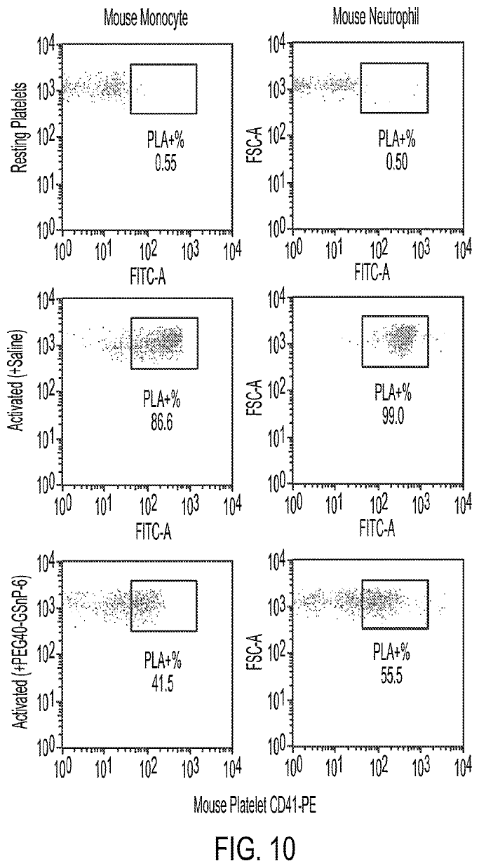

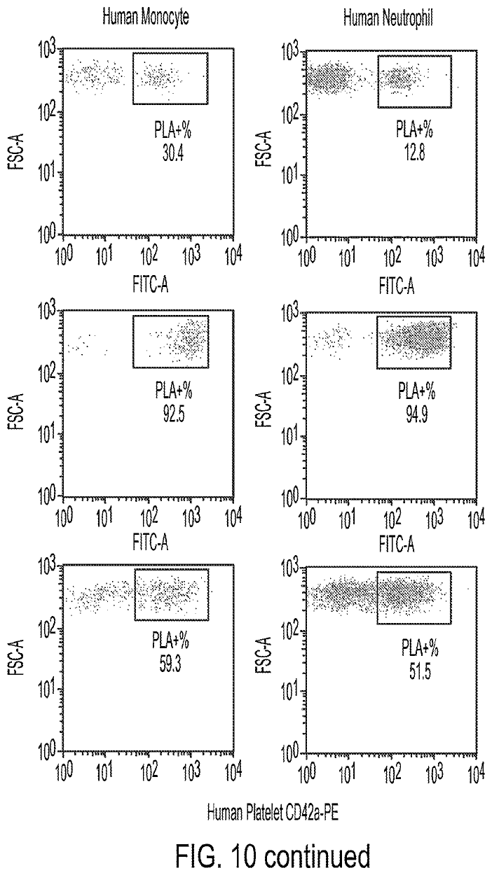

Pharmacokinetics of PG-6. A liquid chromatography tandem mass spectrometry (LC-MS/MS) method was developed to determine the plasma concentration of PG-6. Specifically, low-flow hydrophilic interaction liquid chromatography (HILIC) LC-Orbitrap Fusion Lumos mass spectrometry was used to quantify PG-6 in plasma. A method to distinguish the analyte of interest from other matrix components and identify an abundant precursor-product ion pair was developed to monitor reliably. Optimal analytical performance was achieved using in-source fragmentation to de-PEGylate PG-6 prior to detection (FIG. 2A, FIG. 9). Competing or interfering ions, which co-eluted with GSnP-6 in either plasma controls or plasma isolated from mice injected with saline vehicle, were not detected validating the utility of GSnP-6 as a quantifying peptide (FIG. 10). The full peptide sequence, site-specific sulfation of phenylalanine, site-specific O-glycosylation of threonine, and O-glycan composition of GSnP-6 was confirmed via stepped higher-energy collisional dissociation (HCD) (FIGS. 2B-C). Lability was minimized by setting the RF lens to <30%, maintaining buffered solvents at pH 8.5, and operating in negative ionization mode. A standard curve of PG-6 in pooled plasma was generated with excellent linearity over four orders of magnitude (FIG. 2A). A single intravenous dose of PG-6 was administered to adult mice (n=4/time point). Time-dependent changes in the plasma concentration of PG-6 were consistent with a calculated terminal half-life of 15.65±3.55 hours (FIG. 2D); substantially longer than the reported half-life of several minutes associated with structurally similar non-PEGylated GSP mimics of PSGL-1.^{54,65}

PG-6 inhibits leukocyte/P-selectin binding in a dose dependent manner. Flow cytometry was used to assess the inhibition of P-selectin binding to murine and human leukocytes by GSnP-6 and PG-6. A recombinant mouse P-selectin-Fc chimera (3 μg/mL) was incubated with murine leukocytes along with GSnP-6 or PG-6 (0-120 μM). In a similar manner, a recombinant human P-selectin Fc chimera (3 μg/mL) was incubated with peripheral blood leukocytes from healthy adult volunteers. The binding of P-selectin to leukocytes was measured using a fluorescent labeled anti-Fc antibody, quantified as mean fluorescent intensity, and presented as percent inhibition of binding. PG-6 inhibited P-selectin binding to both mouse and human PMNs and monocytes in a dose-dependent manner with similar inhibition kinetics as observed for GSnP-6 (FIGS. 3A-D).

PG-6 inhibits platelet-leukocyte aggregation in vitro and in vivo. Platelet-leukocyte aggregation is dependent on PSOL-1/P-selectin binding.⁶⁶ In vitro platelet-monocyte and platelet-neutrophil aggregation was evaluated by flow cytometry. Platelet activation was induced by exposure to a PAR4-activating peptide in mouse blood or PAR1-activating peptide in human blood in the presence of PG-6 (120 μM). Murine platelet-monocyte and platelet-neutrophil aggregates were reduced by 45% and 38%, and human platelet-monocyte and platelet-neutrophil aggregates were reduced by 29% and 42%, respectively (FIGS. 4A-B, FIG. 10). Platelet-leukocyte adhesion in vivo was examined using fluorescently labelled platelets following tumor necrosis factor (TNF)-α induction of venular inflammation. PG-6 or saline was administered intravenously (8 μmol/kg) prior to cremaster preparation and platelet microaggregate formation and platelet-neutrophil binding was monitored (n=3-8 mice/group, 7-9 vessels/mouse). PG-6 significantly reduced platelet aggregation as quantified by median integrated fluorescence or normalized fluorescence signal AUC (FIGS. 4C-F).

PG-6 inhibits venous thrombosis without disruption of hemostasis. Administration of PG-6 led to a significant decrease in thrombus formation after electrolytic injury of the murine vena cava; an effect equivalent to that observed for enoxaparin (FIGS. 5A-B). Both treatment with PG-6 and enoxaparin led to a significant reduction in infiltrating Ly6G+ neutrophils and CD68+ macrophages (FIGS. 5C-F). Tail vein transection bleeding time was prolonged after treatment with enoxaparin but was unaffected by administration of PG-6 (FIG. 6).

Discussion

Events mediated by the PSGL-1/P-selectin pathway are central to the pathogenesis of VTE. P-selectin plays a critical role in thrombus initiation, propagation, and the evolution of an attendant inflammatory response, which leads to the release of tissue factor, fibrin deposition, and leukocyte recruitment.^{11,12,24-26} Moreover, clinical studies have shown that elevated plasma P-selectin levels are predictive of VTE in high-risk populations.^{67,68} As such, P-selectin inhibition represents an attractive target to reduce the risk of venous thrombosis. Nonetheless, P-selectin inhibitors have yet to be translated for prevention or treatment of VTE.

Blocking antibodies were the first P-selectin targeted therapies to show efficacy in animal studies of VTE. Wakefield et al. noted a synergistic effect in TNF-α and P-selectin antibody blockade with decreased leukocyte extravasation following vena cava ligation in a rat.²⁹ While the degree of thrombus accumulation was not affected in this model, subsequent studies in primates confirmed both a reduction in thrombus formation and leukocyte extravasation.²⁷ Notably, outcomes in a baboon model varied with underlying venous pathology. For instance, P-selectin antibody blockade led to a 6-fold reduction in thrombus accumulation in segments with pure venous stasis, whereas thrombus persisted in regions associated with underlying endothelial damage due to an occlusive balloon. Early studies of rPSGL-Ig also demonstrated reduced thrombosis in both feline and primate occlusive models of VTE^{28,30,31}, with the treatment effect most marked in regions without underlying endothelial trauma.³⁰

Both the contextual framework and model-dependent treatment effects highlight the inherent variability of existing animal models of VTE.⁶⁹ Depending on the thrombotic stimulus and degree of flow restriction, models differ in degree of hypoxia, endothelial activation and injury,⁷⁰ initial source of tissue factor,^{11,71} thrombus kinetics,⁶² and blood flow.⁷² Further, only baboon and feline models possess venous valves, considered the initial site of VTE in humans.⁷³ As such, defining therapeutic potential in existing animal models of VTE is complicated by a lack of clarity of essential outcomes and translatable pathology. It is noteworthy that clinical trials of P-selectin targeted therapies in VTE have lagged behind its evaluation in other diseases, including acute coronary syndrome, reperfusion injury, or sickle cell vaso-occlusive crisis. To date, the anti-PSGL-1 SelK2 mAb is the only therapy targeting PSGl-1 in the P-selectin/PSGL-1 pathway in ongoing clinical trials. While a number of factors influence the therapeutic development of new chemical entities, it is likely that continued concerns of modest affinity and variable efficacy combined with the aforementioned challenges associated with the commercialization of recombinant therapies have delayed the introduction of P-selectin targeted therapy for VTE.

Among the synthetic inhibitors of P-selectin, PSI-697 and second-generation PSI-421, are best characterized in models of VTE. PSI-697 reduced thrombus weight in mice and decreased walls stiffness, neointima formation, and the extravasation of inflammatory cells in rats.^{33,34} Notably PSI-697 reduced thrombus accumulation in P-selectin- and E-selectin-double-deficient mice, suggesting a potential lack of specificity for selectin signaling. In primate models, both PSI-697 and PSI-421 enhanced vein recanalization and decreased radiologic evidence of inflammation.^{35,36} However PSI-697 failed to inhibit platelet-monocyte aggregation in humans, presumably due to reduced affinity or specificity for P-selectin.⁵⁸ The potential for P-selectin inhibition with oligonucleotide aptamers was demonstrated in a baboon model showing increased venous recanalization and preserved valve competency, but further studies are lacking.³⁷ Moreover, translational challenges persist for aptamer therapy, including rapid renal clearance, enzymatic degradation, endosomal sequestration, and uncertain toxicology.⁷⁴

In these studies, treatment with PG-6 results in a 60% reduction in thrombus weight after 48 hours. Notably, this effect is not associated with increased bleeding time yet is equivalent to that obtained by treatment with enoxaparin. PG-6 reduces both neutrophil and monocyte extravasation at a magnitude similar to enoxaparin treatment. Cellular infiltration of the venous wall is an important measure of the inflammatory component of thrombosis across pre-clinical models.^{11,27-29,32} The electrolytic induced vena cava injury model employed in these studies creates a consistent, partially occlusive thrombus with continuous exposure to venous flow and reliable drug exposure levels. As such, this model is the preferred small animal model for comparing anti-thrombotic agents.⁷²

Enoxaparin has an established history of clinical effectiveness in the treatment of VTE and is the first-line therapy for cancer associated VTE and post-operative prophylaxis.³ Thus, the equivalent anti-thrombotic and anti-inflammatory effects of PG-6 without disruption of hemostasis is a significant finding given the morbidity associated with anticoagulants even at reduced dose regimens. The 15-hour half-life achieved with PEGylation is comparable to current anticoagulants, and is potentially advantageous to the extended half-lives observed with high affinity biologics (crizanlizumab t_{1/2} 10.6 days; rPSGL-Ig t_{1/2} 4 days),^{43,60} whose systemic clearance may require several weeks. Other reported glycopeptide mimics of PSGL-1 possessed a half-life of several minutes, which limits clinical application.⁶⁵

The addition of a PEG moiety is a common strategy to increase drug exposure levels and extend circulating half-life, but the PEGylation of a negatively charged glycopeptide bearing a complex polysaccharide has yet to be reported.⁷⁵ Few if any established protocols exist for either the synthesis or purification of such compounds.⁷⁶ In particular, it was determined that RP-HPLC offered several advantages over anion-exchange chromatography for the purification of PEGylated glycopeptides, including the ability to visualize the progression of the reaction profile, separate GSnP-6 from PG-6 for reuse in the reaction scheme, and eliminate the presence of excess salt. PEGylation can also complicate MS-based peptide analysis. PEGylated peptides exhibit more complexity than traditional drugs of low molecular weight, including broad spectra over a wide m/z range and high molecular weight ions that are outside of the detectable m/z range. To quantify PG-6 concentrations, an LC-MS/MS protocol was established that relied upon in-source fragmentation. In general, mass spectrometry coupled to nanoflow ultra-high-performance liquid chromatography has proven quite versatile in determining the pharmacokinetic profile of a wide range therapeutics due to its wide dynamic range, high sensitivity, analyte specificity, minimal sample preparation, and high throughput capabilities.⁷⁷⁻⁸⁰

These studies have several limitations. First, an electrolytic vena cava injury model of venous thrombosis was employed. While this model reproduces endothelial activation, characteristic of the initiating event of human VTE, it does not recapitulate the role of venous stasis in thrombus initiation. Additionally, while the results reflect thrombus accumulation over 48 hours, this investigation did not evaluate thrombus resolution, venous fibrosis, or valve function. Inhibition was observed for both human and mouse platelet and leukocyte binding assays. It is well documented that plasma P-selectin levels vary with disease and among individuals.⁶⁷ For example, in a post-hoc analysis of the SELECT-CABG trial, initial plasma levels of soluble P-selectin were predictive of a therapeutic effect.⁸¹ As such, plasma P-selectin may serve as an important biomarker of the relative effectiveness of PG-6 inhibition in at-risk individuals. Finally, although tail vein bleeding time is a standard pre-clinical method to assess hemostasis, this model is inexact in predicting clinical bleeding risk.

In summary, blockade of the P-selectin/PSGL-1 pathway by PG-6 inhibited murine and human leukocyte/P-selectin binding in a dose dependent manner and reduced platelet-leukocyte aggregation in vitro and in vivo. PG-6 inhibited venous thrombosis in a pre-clinical model of VTE without impairing hemostasis. These findings support the development of PG-6 as a therapeutic for the prevention and treatment of VTE.

### General Methods

All commercially available reagents and solvents were used without further purification. N,N-Dimethylformamide (227056) and N,N′-diisopropylethylamine (387649) were purchased from Sigma-Aldrich and the PEGylation reagent, 40 kDa methoxy poly(ethylene glycol)-succinimidyl valerate (mPEG-SVA), was purchased from Advanced Biochemicals (MOP2506, PDI 1.06). Reverse phase high performance liquid chromatography (RP-HPLC) was performed using the Waters 2767 Gradient Purification System with Waters 2489 UV/Vis detection module and Waters 2545 Binary Gradient Module equipped with a C18 100 Å (250×30 mm, Phenomenex) column (preparative) or a C18 100 Å (50×4.6 mm, Phenomenex) column (analytical). A Bruker Ultraflex II matrix-assisted laser desorption/ionization time of flight (MALDI-TOF) mass spectrometer was used to analyze samples co-crystallized with super 2,3-dihydroxylbenzoic acid (DHB) matrix. Murine and human protocols were approved by the Institutional Animal Care and Use Committee and Institutional Review Board of Beth Israel Deaconess Medical Center.

### Synthesis of PEG40-GSnP-6 (PG-6)

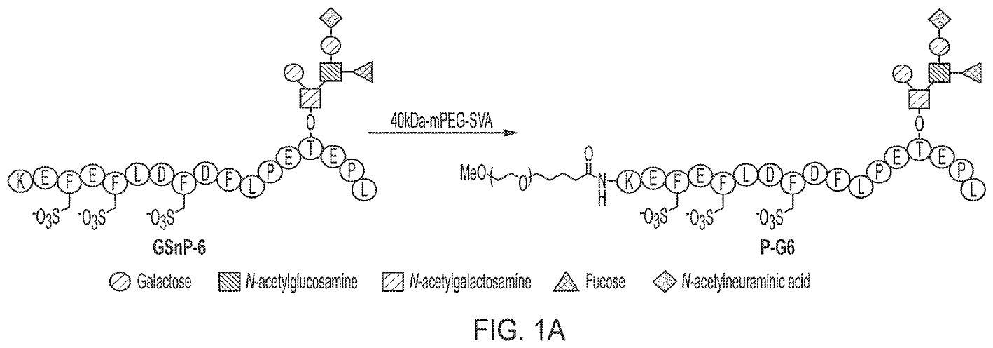

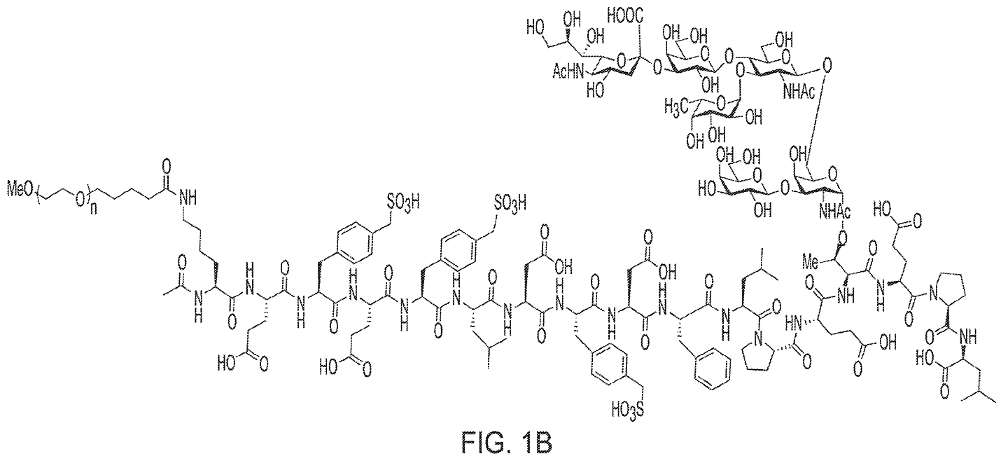

GSnP-6 was synthesized as previously described.⁶¹ PEGylation of GSnP-6 was performed by adding mPEG-SVA (1 eq.) to GSnP-6 (2 eq.) in N,N-dimethylformamide (1.25 mM) at 0° C. Subsequently, N,N′-diisopropylethylamine (DIPEA) was slowly added and shaken for 22 h at 25° C. to a final concentration of GSnP-6 of 20.1 μL/mg. The reaction was quenched using acetic acid and the crude material was purified using RP-HPLC, (preparative) to afford the final product, PG-6 in 62% yield (FIG. 1) and confirmed by MALDI-TOF (FIG. 7). Purity was assessed using RP-HPLC (analytical). RP-HPLC gradient (preparative) using Solvent A—water +0.1% TFA and Solvent B—acetonitrile: 0-10 min, 25% B, 10-40 min, 25-80% B, 40-41 min, 80-98% B, 41-50 min, 98% B, 50-51 min, 98-25% B, 51-60 min, 25% B (PG-6), Rt=20.43 min at a flow rate of 75 mL/min). RP-HPLC gradient (analytical) using Solvent A—water+0.1% TFA, Solvent B—acetonitrile: 0-2 min, 5% B, 2-6 min, 5-98% B, 6-20 min, 98% B, 20-21 min, 98-5% B, 21-22 min, 5% B (PG-6), Rt=4.35 min at a flow rate of 2.5 mL/min) (FIG. 8).

### Pharmacokinetic Profile of PG-6

PG-6 was administered intravenously to C57BL/6 mice at a dose of 8 μmol/kg and blood collected into lithium heparin tubes and centrifuged at 1,500 g for 10 min at room temperature. PG-6 was extracted from 20 μL of plasma by protein precipitation using 80 μL cold methanol and centrifuged at 17,000 g for 20 min at 4° C. The supernatant (80 μL) was dried in vacuo, reconstituted in nanopure water (32 μL), and an aliquot (2 μL) was injected for pre-concentration at a flow rate of 5 μL/min into an UltiMate 3000 RSLCnano ultra-high-performance liquid chromatography (UHPLC) system (Dionex, CA) coupled to an Orbitrap Fusion Lumos mass spectrometer (Thermo Fisher, MA). A binary gradient was applied to an Accucore™ 150 amide hydrophilic interaction liquid chromatography (HILIC) analytical column (150 mm×75 μm ID, 2.6 μm; Thermo Fisher) maintained at 40° C. using solvent (A) comprised of 5 mM ammonium formate in water (pH 8.5) and solvent (B) consisting of 5 mM ammonium formate in 80% (v/v) acetonitrile and 20% (v/v) water (pH 8.5) at a flow rate of 0.4 μL/min: 0-3 min, 99% B: 3-14 min, 99-89% (B); 14-26 min, 89-45% (B); 26-33 min, 45% (B); 33-34 min, 45-99% (B); 34-44 min. 99% (B). An offset voltage (30 V) was applied in the ion source post-UHPLC separation and mass spectrometry (MS) and tandem mass spectrometry (MS/MS) spectra were acquired in the negative mode with the following settings: RF lens, 24%; MS automatic gain control, 5×10⁵; MS maximum injection time, 50 ms; MS resolution, 120,000; MS/MS automatic gain control, 2×10⁵: MS/MS maximum injection time, 200 ms; MS/MS resolution, 30,000; precursor ion isolation width, 1.6 Da; higher-energy collisional dissociation normalized collision energy, 20, 30, 40. Following in-source fragmentation (ISF), m/z 1201.11 (z=3) was used as the quantifying peptide. Peak areas from the extracted chromatograms were used for quantification. An external standard curve was generated by spiking plasma with varying concentrations of PG-6 followed by protein precipitation, as described. A linear regression curve was fitted to the data and used to determine concentrations of PG-6 in collected plasma samples. Compound half-life was determined using a non-compartmental analysis.

### Flow Cytometry

Flow cytometry was used to quantify binding inhibition of P-selectin Fc chimeras to human and mouse leukocytes.⁶¹ Whole mouse or human blood was collected into citrate coated tubes. Leukocytes were isolated by centrifugation and incubated with increasing concentrations of GSnP-6 or PG-6 (0-100 μM) and Fc chimeras of human or mouse P-selectin (R&D Systems, 3 μg/mL) followed by PE-conjugated anti-Fc (1:100, Life Technologies, H10104). The interaction of P-selectin with mouse and human leukocytes was analyzed by flow cytometry (BD LSR II) and quantified (FlowJo) as percent inhibition. Inhibition experiments were conducted in triplicate and representative curves presented.

### Assessment of Platelet-Leukocyte Aggregation In Vitro

Platelet-leukocyte aggregates were quantified in whole blood using dual-label flow cytometry. Anticoagulated human or mouse blood was incubated with 120 μM of PG-6 at room temperature and stimulated with thrombin receptor-activating peptide (human PAR1-activating peptide 40 μM; mouse PAR4-activating peptide 200 μM) to induce platelet P-selectin expression. Two-color flow cytometry was used to quantify platelet-leukocyte aggregates by incubating human samples with anti-CD42a-PE and anti-CD45-APC and mouse samples with anti-CD41-PE and anti-CD45-APC. CD45+monocyte and neutrophil populations were distinguished through characteristic side scatter and quantified as % platelet positive in saline control or PG-6 (120 p M) treated samples.

### Murine Model of Non-Occlusive Venous Thrombosis

Drug efficacy was evaluated in a preclinical mouse model in which a non-occlusive venous thrombosis was induced by electrolytic injury of the inferior vena cava, as detailed elsewhere.⁶² PG-6 was administered intravenously (8 μmol/kg) to male C57BL/6 mice (8-12 weeks of age) immediately prior to electrolytic injury and two hours after injury. Enoxaparin was administered subcutaneously (6 mg/kg) 4 hours prior and 24 hours after electrolytic injury as a clinically relevant control. C57BL/6 mice (Jackson Labs, Bar Harbor, ME, USA) were anesthetized with 2% isoflurane and the inferior vena cava approached via a midline laparotomy. Venous side branches were ligated or cauterized, while posterior branches were left patent. A 25-gauge stainless steel needle, attached to a silver-coated copper wire was inserted into the exposed caudal vena cava and positioned against the anterior wall (anode). A second wire was implanted subcutaneously to complete the circuit (cathode) and a 250 μAmps current applied for 15 min. Subsequently, the needle was removed and a cotton swab held in gentle contact with the puncture site to prevent bleeding. The vena cava and associated thrombus, immediately below the renal veins to just above the bifurcation, was excised 48 hours after injury for determination of wet thrombus weight and histological examination. Specimens were fixed overnight in 10% neutral buffered formalin, processed for paraffin embedding, and 5 μm sections stained with hematoxylin and eosin (H&E) or antibodies specific to neutrophils (Ly-6G, BD Biosciences) and monocytes (CD68, Abeam, Cambridge, MA). The circumference of the vein wall was imaged at 20× magnification and leukocyte infiltration was quantified in 3 representative animals/group, 5 to 10 sections/tissue sample.

### Tail Vein Transection Bleeding Rime

Hemostasis was assessed using a tail vein transection model to determine bleeding time.⁶³ Mice were anesthetized with ketamine and xylazine by intraperitoneal injection and placed on a warming mat at 37° C. Sterile saline, enoxaparin (6 mg/kg), or PG-6 in 125 μL of sterile saline was injected into the penile vein. Five minutes after administration of test compound, the lateral tail vein was transected with a number 11 scalpel blade at a tail width of 2.3 mm and immediately submerged in 37° C. phosphate buffered saline. The bleeding time was determined at that instant when bleeding had ceased for 30 seconds. Animals were excluded if arterial bleeding was present.

### Intravital Microscopy

Recombinant TNF-α (R&D Systems, 0.33 μg) was administered by means of intrascrotal injection 3 hours prior to imaging. Surgical preparation of the mouse cremaster was performed as previously described.64 Mice were anesthetized with an intraperitoneal injection of ketamine HCl (125 mg/kg) and xylazine (12.5 mg/kg) and placed on a 37° C. surgical blanket. The jugular vein was cannulated with PE10 tubing to allow the introduction of reagents, including PG-6 (8 μmol/kg), anti-platelet Dylight 649-anti-CD42b (Emfret, 1 μL/g) and Alexa Fluor 488 anti-Gr-1 (0.1 μL/g) diluted to final volume of 200 μL). The cremaster muscle was exteriorized, immobilized, and superfused with thermocontrolled bicarbonate-buffered saline. Images were obtained using an Olympus AX microscope with 60× water immersion objectives recorded with a Hamamatsu C9300-201/GenIII videoscope with coordinated image acquisition and analysis using SlideBook software (Intelligent Imaging Innovations). For each treatment condition, platelet accumulation was characterized as median integrated fluorescence normalized to vessel area and plotted during a 3-minute interval from 7-9 post capillary venules. The platelet signal was quantified as area under the curve (AUC) for each individual capture and normalized to vessel surface area. Similar levels of adherent leukocytes were observed between all groups.

### Statistical Analysis

Descriptive data are presented as mean±SEM unless otherwise stated. Group comparisons were conducted using one-way ANVOA with Tukey's multiple comparison or Welch's ANOVA with Dunnet's multiple comparison as appropriate for variance of data. Statistical analysis was performed using GraphPad Prism version 8.0.0 for Windows (GraphPad Software, San Diego, CA).

### REFERENCES

1. Benjamin E J, Muntner P, Alonso A, et al. Heart Disease and Stroke Statistics-2019 Update: A Report From the American Heart Association. Vol. 139; 2019.
2. Beckman M G, Hooper W C, Critchley S E, Ortel T L. Venous Thromboembolism. A Public Health Concern. American Journal of Preventive Medicine. 2010; 38(4 SUPPL.):S495-S501.
3. Kearon C, Akl E A, Ornelas J, et al. Antithrombotic therapy for VTE disease: CHEST guideline and expert panel report. Chest. 2016; 149(2):315-352.
4. Carrier M, Abou-Nassar K, Mallick R, et al. Apixaban to prevent venous thromboembolism in patients with cancer. New England Journal of Medicine. 2019:380(8):711-719.
5. Coulis A A. Mackey W C. A Review of the Efficacy and Safety Profiles of the Novel Oral Anticoagulants in the Treatment and Prevention of Venous Thromboembolism. Clinical Therapeutics. 2018; 40(12):2140-2167.
6. Aryal M R, Gosain R, Donato A, et al. Systematic review and meta-analysis of the efficacy and safety of apixaban compared to rivaroxaban in acute VTE in the real world. Blood Advances. 2019; 3(15):2381-2387.
7. Ay C, Beyer-Westendorf J, Pabinger I. Treatment of cancer-associated venous thromboembolism in the age of direct oral anticoagulants. Annals of Oncology. 2019; 30(6):897-907.
8. Riva N, Donadini M P, Ageno W. Epidemiology and pathophysiology of venous thromboembolism: Similarities with atherothrombosis and the role of inflammation. Thrombosis and Haemostasis. 2015:113(6):1176-1183.
9. Bacani A K, Gabriel S E, Crowson C S, Heit J A, Matteson E L. Noncardiac vascular disease in rheumatoid arthritis: Increase in venous thromboembolic events? Arthritis and Rheumatism. 2012; 64(1):53-61.
10. Grainge M J, West J, Card T R. Venous thromboembolism during active disease and remission in inflammatory bowel disease: a cohort study. The Lancet. 2010; 375(9715):657-663.
11. von Bruhl M L, Stark K, Steinhart A, et al. Monocytes, neutrophils, and platelets cooperate to initiate and propagate venous thrombosis in mice in vivo. J Exp Med. 2012:209(4):819-835.
12. Falati S, Liu Q, Gross P, et al. Accumulation of tissue factor into developing thrombi in vivo is dependent upon microparticle P-selectin glycoprotein ligand 1 and platelet P-selectin. The Journal of experimental medicine. 2003; 197(11):1585-1598.
13. Darbousset R, Thomas G M, Mezouar S, et al. Tissue factor-positive neutrophils bind to injured endothelial wall and initiate thrombus formation. Blood. 2012; 120(10):2133-2143.
14. Clark S R, Ma A C, Tavener S A, et al. Platelet TLR4 activates neutrophil extracellular traps to ensnare bacteria in septic blood. Nature Medicine. 2007:13(4):463-469.
15. Ammollo C T, Semeraro F, Xu J, Esmon N L, Esmon C T. Extracellular histones increase plasma thrombin generation by impairing thrombomodulin-dependent protein C activation. J Thromb Haemost. 2011; 9(9):1795-1803.
16. Fuchs T A, Brill A, Duerschmied D, et al. Extracellular DNA traps promote thrombosis. Proc Natl Acad Sci USA. 2010; 107(36):15880-15885.
17. Ward C M, Tetaz T J, Andrews R K, Berndt M C. Binding of the von Willebrand factor A1 domain to histone. Thromb Res. 1997; 86(6):469-477.
18. Massberg S, Grahl L, von Bruehl M L, et al. Reciprocal coupling of coagulation and innate immunity via neutrophil serine proteases. Nat Med. 2010:16(8):887-896.
19. Larsen E, Celi A, Gilbert G E, et al. PADGEM protein: A receptor that mediates the interaction of activated platelets with neutrophils and monocytes. Cell. 1989; 59(2):305-312.
20. McEver R P, Beckstead J H, Moore K L, Marshall-Carlson L, Bainton D F. GMP-140, a platelet α-granule membrane protein, is also synthesized by vascular endothelial cells and is localized in Weibel-Palade bodies. Journal of Clinical Investigation. 1989; 84(1):92-99.
21. Dore M, Korthuis R J, Granger D N, Entman M L, Smith C W. P-selectin mediates spontaneous leukocyte rolling in vivo. Blood. 1993; 82(4):1308-1316.
22. Zarbock A, Ley K, McEver R P, Hidalgo A. Leukocyte ligands for endothelial selectins: Specialized glycoconjugates that mediate rolling and signaling under flow. Blood. 2011; 118(26):6743-6751.
23. Mayadas T N, Johnson R C, Rayburn H, Hynes R O, Wagner D D. Leukocyte rolling and extravasation are severely compromised in P selectin-deficient mice. Cell. 1993; 74(3):541-554.
24. Palabrica T, Lobb R, Furie B C, et al. Leukocyte accumulation promoting fibrin deposition is mediated in vivo by P-selectin on adherent platelets. Nature. 1992; 359(6398):848-851.
25. Sullivan V V, Hawley A E, Farris D M, et al. Decrease in fibrin content of venous thrombi in selectin-deficient mice. J Surg Res. 2003:109(1):1-7.
26. Myers D, Farris D, Hawley A, et al. Selectins influence thrombosis in a mouse model of experimental deep venous thrombosis. Journal of Surgical Research. 2002; 108(2):212-221.
27. Downing L J, Wakefield T W, Strieter R M, et al. Anti-P-selectin antibody decreases inflammation and thrombus formation in venous thrombosis. Journal of Vascular Surgery. 1997; 25(5):816-828.
28. Myers D D, Hawley A E, Farris D M, et al. P-selectin and leukocyte microparticles are associated with venous thrombogenesis. Journal of Vascular Surgery. 2003; 38(5):1075-1089.
29. Wakefield T W, Strieter R M, Downing I J, et al. P-selectin and TNF inhibition reduce venous thrombosis inflammation. Journal of Surgical Research. 1996; 64(1):26-31.
30. Eppihimer M J, Schaub R G. P-selectin-dependent inhibition of thrombosis during venous stasis. Arteriosclerosis, Thrombosis, and Vascular Biology. 2000; 20(11):2483-2488.
31. Wakefield T W, Strieter R M, Schaub R, et al. Venous thrombosis prophylaxis by inflammatory inhibition without anticoagulation therapy. Journal of Vascular Surgery. 2000:31(2):309-324.
32. Thanaporn P, Myers D D, Wrobleski S K, et al. P-selectin inhibition decreases post-thrombotic vein wall fibrosis in a rat model. Surgery. 2003; 134(2):365-371.
33. Myers D D, Rectenwald J E, Bedard P W, et al. Decreased venous thrombosis with an oral inhibitor of P selectin. Journal of Vascular Surgery. 2005; 42(2):329-336.
34. Myers D D, Henke P K, Bedard P W, et al. Treatment with an oral small molecule inhibitor of P selectin (PSI-697) decreases vein wall injury in a rat stenosis model of venous thrombosis. Journal of Vascular Surgery. 2006; 44(3):625-632.
35. Myers D D, Wrobleski S K, Longo C, et al. Resolution of venous thrombosis using a novel oral small-molecule inhibitor of P-selectin (PSI-697) without anticoagulation.

Thrombosis and haemostasis. 2007; 97(3):400-407.
36. Meier T R, Myers D D, Wrobleski S K, et al. Prophylactic P-selectin inhibition with PSI-421 promotes resolution of venous thrombosis without anticoagulation. Thrombosis and Haemostasis. 2008; 99(02):343-351.
37. Diaz J A, Wrobleski S K, Alvarado C M, et al. P-Selectin inhibition therapeutically promotes thrombus resolution and prevents vein wall fibrosis better than enoxaparin and an inhibitor to von willebrand factor. Arteriosclerosis, Thrombosis, and Vascular Biology. 2015; 35(4):829-837.
38. Prado M S, Bendtzen K, Andrade L E C. Biological anti-TNF drugs: immunogenicity underlying treatment failure and adverse events. Expert opinion on drug metabolism & toxicology. 2017; 13(9):985-995.
39. Harding F A, Stickler M M, Razo J, DuBridge R B. The immunogenicity of humanized and fully human antibodies: Residual immunogenicity resides in the CDR regions, mAbs. 2010:2(3):256-265.
40. Solá R J, Griebenow K. Glycosylation of Therapeutic Proteins. BioDrugs. 2010:24(1):9-21.
41. Mertens P, Maes A, Nuyts J, et al. Recombinant P-selectin glycoprotein ligand-immunoglobulin, a P-selectin antagonist, as an adjunct to thrombolysis in acute myocardial infarction. The P-Selectin Antagonist Limiting Myonecrosis (PSALM) trial. American Heart Journal. 2006:152(1):125.e121-125.e128.
42. Busuttil R W, Lipshutz G S, Kupiec-Weglinski J W, et al. RPSGL-Ig for improvement of early liver allograft function: A double-blind, placebo-controlled, single-center phase II study. American Journal of Transplantation. 2011; 11(4):786-797.
43. Osama Gaber A, Mulgaonkar S, Kahan B D, et al. YSPSL (rPSGL-Ig) for improvement of early renal allograft function: A double-blind, placebo-controlled, multi-center Phase IIa study. Clinical Transplantation. 2011; 25(4):523-533.
44. Arnold D F, Misbah S A. Cetuximab-induced anaphylaxis and IgE specific for galactose-α-1,3-galactose. New England Journal of Medicine. 2008:358(25):2735-2736.
45. Jefferis R. Glycosylation as a strategy to improve antibody-based therapeutics. Nature Reviews Drug Discovery. 2009:8(3):226-234.
46. Hossler P. Khattak S F, Li Z J. Optimal and consistent protein glycosylation in mammalian cell culture. Glycobiology. 2009:19(9):936-949.
47. Patel M S. Miranda-Nieves D, Chen J, Haller C A, Chaikof E L. Targeting P-selectin glycoprotein ligand-1/P-selectin interactions as a novel therapy for metabolic syndrome.

Translational Research. 2017; 183:1-13.
48. Chang J, Patton J T, Sarkar A, Ernst B, Magnani J L, Frenette P S. GMI-1070, a novel pan-selectin antagonist, reverses acute vascular occlusions in sickle cell mice. Blood. 2010:116(10):1779-1786.
49. Huang A, Moretto A, Janz K, et al. Discovery of 2-[1-(4-chlorophenyl)cyclopropyl]-3-hydroxy-8-(trifluoromethyl)quinoline-4-carboxylic acid (PSI-421), a P-selectin inhibitor with improved pharmacokinetic properties and oral efficacy in models of vascular injury. J Med Chem. 2010; 53(16):6003-6017.
50. Kaila N, Janz K, DeBernardo S, et al. Synthesis and biological evaluation of quinoline salicylic acids as P-selectin antagonists. J Med Chem. 2007; 50(1):21-39.
51. Kranich R, Busemann A S, Bock D, et al. Rational design of novel, potent small molecule pan-selectin antagonists. J Med Chem. 2007:50(6):1101-1115.
52. Watz H, Bock D, Meyer M, et al. Inhaled pan-selectin antagonist Bimosiamose attenuates airway inflammation in COPD. Pulm Pharmacol Ther. 2013:26(2):265-270.
53. Leppänen A, Yago T, McEver R P, Cummings R D. Model glycosulfopeptides from P-selectin glycoprotein ligand-1 require tyrosine sulfation and a core 2-branched O-glycan to bind to L-selectin. J Biol Chem. 2003:278(29):26391-26400.
54. Leppänen A, Mehta P, Ouyang Y B, et al. A novel glycosulfopeptide binds to P-selectin and inhibits leukocyte adhesion to P-selectin. Journal of Biological Chemistry. 1999:274(35):24838-24848.
55. Leppanen A, White S P, Helin J, McEver R P, Cummings R D. Binding of glycosulfopeptides to P-selectin requires stereospecific contributions of individual tyrosine sulfate and sugar residues. J Biol Chem. 2000; 275(50):39569-39578.
56. Ushiyama S, Laue™, Moore K L, Erickson H P, McEver R P. Structural and functional characterization of monomeric soluble P-selectin and comparison with membrane P-selectin. Journal of Biological Chemistry. 1993; 268(20):15229-15237.
57. Mehta P, Cummings R D, McEver R P. Affinity and kinetic analysis of P-selectin binding to P-selectin glycoprotein ligand-1. Journal of Biological Chemistry. 1998; 273(49):32506-32513.
58. Japp A G, Chelliah R, Tattersall L, et al. Effect of PSI-697, a novel P-selectin inhibitor, on platelet-monocyte aggregate formation in humans. Journal of the American Heart Association. 2013; 2(1):1-6.
59. M. J T, T W, T. L M, et al. Randomized phase 2 study of GMI-1070 in SCD: Reduction in time to resolution of vaso-occlusive events and decreased opioid use. Blood. 2015:125(17):2656-2664.
60. Research FDACfDEa. Multi-Discipline Review: ADAKVEO (Crizanlizumab-tmca): 2019.
61. Krishnamurthy V R, Sardar M Y R, Ying Y, et al. Glycopeptide analogues of PSGL-1 inhibit P-selectin in vitro and in vivo. Nature Communications. 2015; 6.
62. Diaz J A, Alvarado C M, Wrobleski S K, et al. The electrolytic inferior vena cava model (EIM) to study thrombogenesis and thrombus resolution with continuous blood flow in the mouse. Thrombosis and Haemostasis. 2013; 109(06):1158-1169.
63. Culmer D L, Dunbar M L, Hawley A E, et al. E-selectin inhibition with GMI-1271 decreases venous thrombosis without profoundly affecting tail vein bleeding in a mouse model. Thrombosis and Haemostasis. 2017; 117(6):1171-1781.
64. Kim KHo, Barazia A, Cho J. Real-time imaging of heterotypic platelet-neutrophil interactions on the activated endothelium during vascular inflammation and thrombus Formation in live mice. Journal of visualized experiments: JoVE. 2013(74):1-7.
65. Hicks A E R, Leppanen A, Cummings R D, McEver R P, Hellewell P G, Norman K E. Glycosulfopeptides modeled on P-selectin glycoprotein ligand 1 inhibit P-selectin-dependent leukocyte rolling in vivo. The FASEB journal: official publication of the Federation of American Societies for Experimental Biology. 2002; 16(11):1461-1462.
66. Evangelista V, Manarini S, Sideri R, et al. Platelet/Polymorphonuclear Leukocyte Interaction: P-Selectin Triggers Protein-Tyrosine Phosphorylation-Dependent CD11b/CD18 Adhesion: Role of PSGL-1 as a Signaling Molecule. Blood. 1999; 93(3):876-885.
67. Ay C, Jungbauer L V, Sailer T, et al. High concentrations of soluble P-selectin are associated with risk of venous thromboembolism and the P-selectin Thr715 variant. Clin Chem. 2007; 53(7):1235-1243.
68. Ay C, Simanek R, Vormittag R, et al. High plasma levels of soluble P-selectin are predictive of venous thromboembolism in cancer patients: Results from the Vienna Cancer and Thrombosis Study (CATS). Blood. 2008:112(7):2703-2708.
69. Diaz J A, Obi A T. Myers Jr D D, et al. Critical review of mouse models of venous thrombosis. Arterioscler Thromb Vasc Biol. 2012:32(3):556-562.
70. Budnik I, Brill A. Immune Factors in Deep Vein Thrombosis Initiation. Trends in Immunology. 2018; 39(8):610-623.
71. Zhou J, May L, Liao P, Gross P L, Weitz J I. Inferior vena cava ligation rapidly induces tissue factor expression and venous thrombosis in rats. Arteriosclerosis, Thrombosis, and Vascular Biology. 2009; 29(6):863-869.
72. Diaz J A, Saha P, Cooley B, et al. Choosing a mouse model of venous thrombosis: a consensus assessment of utility and application. J Thromb Haemost. 2019; 17(4):699-707.
73. Brooks E G, Trotman W, Wadsworth M P, et al. Valves of the deep venous system: An overlooked risk factor. Blood. 2009:114(6):1276-1279.
74. Dowdy S F. Overcoming cellular barriers for RNA therapeutics. Nature Biotechnology. 2017; 35(3):222-229.
75. Bossard M, Vicent M. Polymer-protein conjugates: Elsevier: 2020.
76. Greenwald R B. PEG drugs: An overview. Journal of Controlled Release. 2001; 74(1-3):159-171.
77. de Moraes N V, Moretti R A C, Furr E B, McCurdy C R, Lanchote V L. Determination of mitragynine in rat plasma by LC-MS/MS: Application to pharmacokinetics. Journal of Chromatography B: Analytical Technologies in the Biomedical and Life Sciences. 2009:877(24):2593-2597.
78. Fernández Ocafia M, James I T, Kabir M. et al. Clinical pharmacokinetic assessment of an anti-MAdCAM monoclonal antibody therapeutic by LC-MS/MS. Analytical Chemistry. 2012; 84(14):5959-5967.
79. Chang L, Ren Y, Cao L, et al. Simultaneous determination and pharmacokinetic study of six flavonoids from Fructus Sophorae extract in rat plasma by LC-MS/MS. Journal of Chromatography B: Analytical Technologies in the Biomedical and Life Sciences. 2012; 904:59-64.
80. Wring S A, Randolph R, Park S H, et al. Preclinical pharmacokinetics and pharmacodynamic target of SCY-078, a first-in-class orally active antifungal glucan synthesis inhibitor, in murine models of disseminated candidiasis. Antimicrobial Agents and Chemotherapy. 2017; 61(4):1-15.
81. Stähli B E, Tardif J-C, Carrier M, et al. Effects of P-Selectin Antagonist Inclacumab in Patients Undergoing Coronary Artery Bypass Graft Surgery. Journal of the American College of Cardiology. 2016; 67(3):344-346.

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A glycopeptide comprising the formula Y¹X¹Y²X²X³Y³X⁴X⁵X⁶Z¹X⁷W¹ (SEQ ID NO: 1) or a salt thereof, wherein:
W¹ is threonine or serine substituted with a saccharide or polysaccharide,
X¹, X², X³, X¹, X⁵, X⁶, and X⁷ are each individually and independently any amino acid,
Y¹, Y², and Y³ are each individually and independently tyrosine, phenylalanine, or phenylglycine, and wherein Y¹, Y², and Y³ are each independently unsubstituted or substituted with —SO₃H, —CH₂SO₃H, —CF₂SO₃H, —CO₂H, —CONH₂, —NHSO₂CH₃, —SO₂NH₂, or —CH₂PO₃H;
wherein at least one of Y¹, Y², and Y³ is substituted with —CH₂SO₃H;
Z¹ is proline or hydroxyproline; and
at least one amino acid residue is substituted with -L¹-PEG;
wherein L¹ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, or combinations thereof; and
PEG is linear or branched polyethylene glycol.

2. The glycopeptide of claim 1, or a salt thereof, wherein Y¹, Y², and Y³ are substituted with —CH₂SO₃H.

3. The glycopeptide of claim 1 or 2, or a salt thereof, wherein X¹, X³, X⁴, and X⁷ are each individually and independently E, D, N, or Q.

4. The glycopeptide of any one of the preceding claims, or a salt thereof, wherein X², X⁵, and X⁶ are each individually and independently L, F, V, A, or I.

5. The glycopeptide of any one of claims 1-4, or a salt thereof, wherein the isolated glycopeptide comprises
(SEQ ID NO: 2)
Y¹EY¹LDY³DFLZ¹EW¹,
(SEQ ID NO: 3)
Y¹EY²LDY³DFLZ¹EW¹EP,
(SEQ ID NO: 4)
Y¹EY²LDY³DFLZ¹EW¹EPL,
(SEQ ID NO: 5)
EY¹EY²LDY³DFLZ¹EW¹,
(SEQ ID NO: 6)
EY¹EY²LDY³DFLZ¹EW¹E,
(SEQ ID NO: 7)
EY¹EY²LDY³DFLZ¹EW¹EP,
(SEQ ID NO: 8)
EYEY²LDY³DFLZ¹EW¹EPL,
(SEQ ID NO: 9)
KEY¹EY²LDY³DFLZ¹EW¹,
(SEQ ID NO: 10)
KEY¹EY²LDY³DFLZ¹EW¹E,
(SEQ ID NO: 11)
KEY¹EY²LDY³DFLZ¹EW¹EP,
or
(SEQ ID NO: 12)
KEY¹EY²LDY³DFLZ¹EW¹EPL.

6. The glycopeptide of any one of the preceding claims, or a salt thereof, wherein the saccharide or polysaccharide comprises one or more sugars selected from the group consisting of: galactose, fucose, 2-(acetylamino)-2-deoxy-galactose, 2-(acetylamino)-2-deoxy-glucose, and 5-acetamido-3,5-dideoxy-glycero-galacto-2-nonulosonic acid.

7. The glycopeptide of any one of the preceding claims, or a salt thereof, wherein the polysaccharide comprises 2-(acetylamino)-2-deoxy-galactose alpha 1 bonded to W¹, a first galactose beta 3 bonded to 2-(acetylamino)-2-deoxy-galactose, 2-(acetylamino)-2-deoxy-glucose beta 6 bonded to 2-(acetylamino)-2-deoxy-galactose, fucose alpha 3 bonded to 2-(acetylamino)-2-deoxyglucose, a second galactose beta 4 bonded to 2-(acetylamino)-2-deoxy-glucose, and 5-acetamido-3,5-dideoxy-glycero-galacto-2-nonulosonic acid alpha 3 bonded to the second galactose.

8. The glycopeptide of any one of claims 1-6, or a salt thereof, wherein the polysaccharide is sialyl Lewis X or sialyl Lewis A.

9. The glycopeptide of any of claims 1-7, or a salt thereof, comprising the formula KEY¹EY²LDY³DFLZ¹EW¹EPL (SEQ ID NO: 12), wherein:
W¹ is threonine substituted with a polysaccharide,
Y¹, Y², and Y³ are phenylalanine 4-substituted with —CH₂SO₃H,
Z¹ is proline,
the polysaccharide comprises 2-(acetylamino)-2-deoxy-galactose alpha 1 bonded to W¹, a first galactose beta 3 bonded to 2-(acetylamino)-2-deoxy-galactose, 2-(acetylamino)-2-deoxy-glucose beta 6 bonded to 2-(acetylamino)-2-deoxy-galactose, fucose alpha 3 bonded to 2-(acetylamino)-2-deoxyglucose, a second galactose beta 4 bonded to 2-(acetylamino)-2-deoxy-glucose, and 5-acetamido-3,5-dideoxy-glycero-galacto-2-nonulosonic acid alpha 3 bonded to the first galactose; and
at least one amino acid residue is substituted with -L¹-PEG.

10. The glycopeptide of claim 9, wherein at least one instance of K, E, or D is substituted with -L¹-PEG.

11. The glycopeptide of claim 10, wherein K is substituted with -L¹-PEG.

12. The glycopeptide of any of the preceding claims, or a salt thereof, wherein the PEG comprises 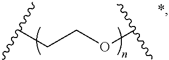
wherein “*” is attached to L¹ and n is an integer between 20 and 3,500, inclusive.

13. The glycopeptide of any one of the preceding claims, or a salt thereof, wherein the PEG terminates in one or more hydroxy or alkoxy end groups.

14. The glycopeptide of any one of the preceding claims, or a salt thereof, wherein L¹ has the structure: 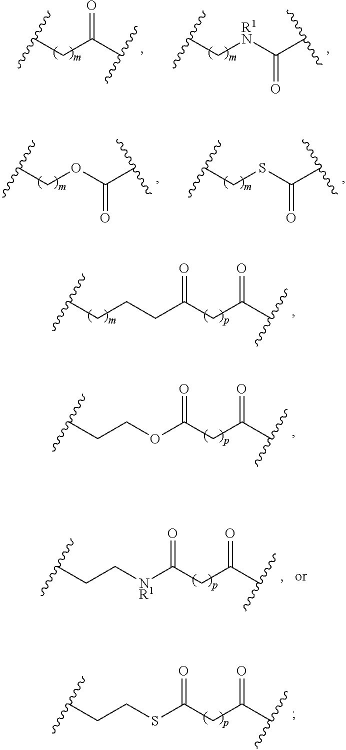
wherein
m is 0 to 20:
p is 1 to 20; and
R¹ is hydrogen or alkyl.

15. The glycopeptide of claim 14, or a salt thereof, wherein L¹ has the structure: 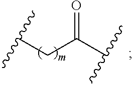
wherein m is 4.

16. The glycopeptide of any one of claims 1-13, or a salt thereof, wherein L¹ has the structure: 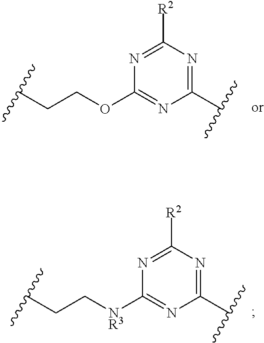
wherein
R¹ is hydrogen or alkyl; and
R² is —H, a halogen, —OR³, —N(R³)₂, —SR³, or —C(R³)₃, wherein R³ is —H, substituted or unsubstituted aliphatic, substituted or unsubstituted heteroaliphatic, substituted or unsubstituted aryl, substituted or unsubstituted heteroaryl, substituted or unsubstituted carbocyclic, or substituted or unsubstituted heterocyclic, or combinations thereof.

17. The glycopeptide of any one of claims 1-13, or a salt thereof, wherein L¹ has the structure: 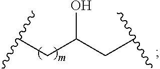
wherein m is 0 to 20.

18. The glycopeptide of any one of claims 1-13, or a salt thereof, wherein L¹ has the structure: 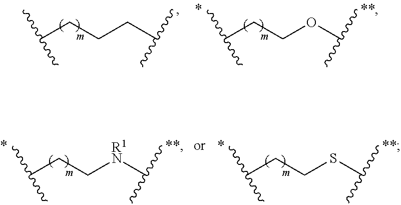
wherein
m is 0 to 20;
R¹ is hydrogen or alkyl;
“*” is attached to the PEG; and
“**” is attached to the glycopeptide.

19. The glycopeptide of any one of claims 1-13, or a salt thereof, wherein L¹ has the structure: 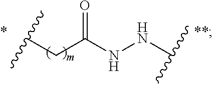
wherein
m is 0 to 20;
“*” is attached to the PEG; and
“**” is attached to the glycopeptide.

20. The glycopeptide of any one of claims 1-7 or 9-15, or a salt thereof, comprising the formula
KEY¹EY²LDY³DFLZ¹EW¹EPL (SEQ ID NO: 12), wherein:
W¹ is threonine substituted with a polysaccharide,
Y¹, Y², and Y³ are phenylalanine 4-substituted with —CH₂SO₃H,
Z¹ is proline,
the polysaccharide comprises 2-(acetylamino)-2-deoxy-galactose alpha 1 bonded to W¹, a first galactose beta 3 bonded to 2-(acetylamino)-2-deoxy-galactose, 2-(acetylamino)-2-deoxy-glucose beta 6 bonded to 2-(acetylamino)-2-deoxy-galactose, fucose alpha 3 bonded to 2-(acetylamino)-2-deoxyglucose, a second galactose beta 4 bonded to 2-(acetylamino)-2-deoxy-glucose, and 5-acetamido-3,5-dideoxy-glycero-galacto-2-nonulosonic acid alpha 3 bonded to the first galactose,
K is substituted with -L¹-PEG, wherein
the PEG is linear and terminates in —OCH₃; and
L¹ has the structure 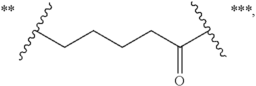 [SMILES: *CCCCC(*)=O]
wherein “**” is attached to PEG, and “***” is attached to K.

21. The glycopeptide of any one of claims 1-7 or 9-15, or a salt thereof, comprising the formula
KEY¹EY²LDY³DFLZ¹EW¹EPL (SEQ ID NO: 12), wherein:
W¹ is threonine substituted with a polysaccharide,
Y¹, Y², and Y³ are phenylalanine 4-substituted with —CH₂SO₃H,
Z¹ is proline,
the polysaccharide comprises 2-(acetylamino)-2-deoxy-galactose alpha 1 bonded to W¹, a first galactose beta 3 bonded to 2-(acetylamino)-2-deoxy-galactose, 2-(acetylamino)-2-deoxy-glucose beta 6 bonded to 2-(acetylamino)-2-deoxy-galactose, fucose alpha 3 bonded to 2-(acetylamino)-2-deoxyglucose, a second galactose beta 4 bonded to 2-(acetylamino)-2-deoxy-glucose, and 5-acetamido-3,5-dideoxy-glycero-galacto-2-nonulosonic acid alpha 3 bonded to the first galactose,
K is substituted with -L¹-PEG, wherein
the PEG is branched and terminates in —OCH₃; and
L¹ has the structure 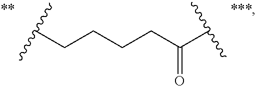 [SMILES: *CCCCC(*)=O]
wherein “**” is attached to PEG, and “***” is attached to K.

22. The glycopeptide, or salt thereof, of any one of claims 1-21, characterized as having similar efficacy to that of the corresponding non-PEGylated glycopeptide.

23. The glycopeptide, or salt thereof, of any one of claims 1-22, characterized as having an IC₅₀ for P-selectin that is similar to the IC₅₀ for P-selectin of the corresponding non-PEGylated glycopeptide.

24. A pharmaceutical composition comprising a glycopeptide of any one of claims 1-23, or a salt thereof, and a pharmaceutically acceptable excipient.

25. The pharmaceutical composition of claim 24, wherein the composition is in the form of a pill, tablet, capsule, or gel.

26. The pharmaceutical composition of claim 24, wherein the composition is in the form of an aqueous saline buffer.

27. The pharmaceutical composition of any one of claims 24-26, wherein the pharmaceutically acceptable excipient is a saccharide or polysaccharide.

28. A method of treating or preventing cardiovascular disease, atherosclerosis, atherosclerotic lesions, thrombus formation, thromboembolism, stroke, myocardial infarction, or cancer comprising administering an effective amount of a glycopeptide of any one of claims 1-23, or a salt thereof, or a pharmaceutical composition of claims 24-27, to a subject in need thereof.

29. The method of claim 28, wherein the subject is at risk of, exhibiting symptoms of, or diagnosed with atherosclerosis, atherosclerotic lesions, thrombus formation, thromboembolism, stroke, myocardial infarction, or cancer.

30. A method of treating or preventing allergy or lung diseases comprising administering an effective amount of a glycopeptide of any one of claims 1-23, or a salt thereof, or a pharmaceutical composition of claims 24-27, to a subject in need thereof.

31. The method of claim 30, wherein the subject is at risk of, exhibiting symptoms of, or diagnosed with asthma, bronchitis, emphysema, and chronic obstructive pulmonary disease (COPD).

32. The method of any one of claims 28-31, wherein the glycopeptide, or salt thereof, has similar efficacy to that of the corresponding non-PEGylated glycopeptide.

33. The method of any one of claims 28-32, wherein the glycopeptide, or salt thereof, has a similar IC₅₀ for a protein as the corresponding non-PEGylated glycopeptide.

34. The method of claim 33, wherein the protein is P-selectin.

35. The method of any one of claims 28-34, wherein the glycopeptide, or salt thereof, has an IC₅₀ for P-selectin that is similar to the IC₅₀ for P-selectin of the corresponding non-PEGylated glycopeptide.

36. A method of inhibiting P-selectin comprising contacting a cell, tissue, or biological sample with an effective amount of a glycopeptide of any one of claims 1-23, or a salt thereof, or a pharmaceutical composition of claims 24-27.

37. The method of claim 36, wherein the glycopeptide, or salt thereof, binds P-selectin.

38. The method of claim 36 or 37, wherein the cell is a human monocyte.

39. The method of claim 36 or 37, wherein the cell is a human neutrophil.

40. The method of claim 36 or 37, wherein the cell is a mouse monocyte.

41. The method of claim 36 or 37, wherein the cell is a mouse neutrophil.


SEQUENCE LISTING (12 sequences)

SEQ ID NO: 1 - protein, 12 aa, Artificial Sequence
  Synthetic
  mod_res (1)..(1): X may be tyrosine, phenylalanine, or phenylglycine
  misc_feature (2)..(2): Xaa can be any naturally occurring amino acid
  mod_res (3)..(3): X may be tyrosine, phenylalanine, or phenylglycine
  misc_feature (4)..(5): Xaa can be any naturally occurring amino acid
  mod_res (6)..(6): X may be tyrosine, phenylalanine, or phenylglycine
  misc_feature (7)..(9): Xaa can be any naturally occurring amino acid
  mod_res (10)..(10): X may be proline or hydroxyproline
  misc_feature (11)..(11): Xaa can be any naturally occurring amino acid
  mod_res (12)..(12): X may be threonine or serine substituted with a saccharide or polysaccharide
XXXXXXXXXXXX

SEQ ID NO: 2 - protein, 15 aa, Artificial Sequence
  Synthetic
  mod_res (1)..(1): X may be phenylalanine 4
  mod_res (3)..(3): X may be phenylalanine 4
  mod_res (6)..(6): X may be phenylalanine 4
  mod_res (10)..(10): X may be proline
  mod_res (12)..(12): X may be threonine substituted with a polysaccharide
XEXLDXDFLXEXEPL

SEQ ID NO: 3 - protein, 14 aa, Artificial Sequence
  Synthetic
  mod_res (1)..(1): X may be phenylalanine 4
  mod_res (3)..(3): X may be phenylalanine 4
  mod_res (6)..(6): X may be phenylalanine 4
  mod_res (10)..(10): X may be proline
  mod_res (12)..(12): X may be threonine substituted with a polysaccharide
XEXLDXDFLXEXEP

SEQ ID NO: 4 - protein, 15 aa, Artificial Sequence
  Synthetic
  mod_res (1)..(1): X may be phenylalanine 4
  mod_res (3)..(3): X may be phenylalanine 4
  mod_res (6)..(6): X may be phenylalanine 4
  mod_res (10)..(10): X may be proline
  mod_res (12)..(12): X may be threonine substituted with a polysaccharide
XEXLDXDFLXEXEPL

SEQ ID NO: 5 - protein, 13 aa, Artificial Sequence
  Synthetic
  mod_res (2)..(2): X may be phenylalanine 4
  mod_res (4)..(4): X may be phenylalanine 4
  mod_res (7)..(7): X may be phenylalanine 4
  mod_res (11)..(11): X may be proline
  mod_res (13)..(13): X may be threonine substituted with a polysaccharide
EXEXLDXDFLXEX

SEQ ID NO: 6 - protein, 14 aa, Artificial Sequence
  Synthetic
  mod_res (2)..(2): X may be phenylalanine 4
  mod_res (4)..(4): X may be phenylalanine 4
  mod_res (7)..(7): X may be phenylalanine 4
  mod_res (11)..(11): X may be proline
  mod_res (13)..(13): X may be threonine substituted with a polysaccharide
EXEXLDXDFLXEXE

SEQ ID NO: 7 - protein, 15 aa, Artificial Sequence
  Synthetic
  mod_res (2)..(2): X may be phenylalanine 4
  mod_res (4)..(4): X may be phenylalanine 4
  mod_res (7)..(7): X may be phenylalanine 4
  mod_res (11)..(11): X may be proline
  mod_res (13)..(13): X may be threonine substituted with a polysaccharide
EXEXLDXDFLXEXEP

SEQ ID NO: 8 - protein, 16 aa, Artificial Sequence
  Synthetic
  mod_res (2)..(2): X may be phenylalanine 4
  mod_res (4)..(4): X may be phenylalanine 4
  mod_res (7)..(7): X may be phenylalanine 4
  mod_res (11)..(11): X may be proline
  mod_res (13)..(13): X may be threonine substituted with a polysaccharide
EXEXLDXDFLXEXEPL

SEQ ID NO: 9 - protein, 14 aa, Artificial Sequence
  Synthetic
  mod_res (3)..(3): X may be phenylalanine 4
  mod_res (5)..(5): X may be phenylalanine 4
  mod_res (8)..(8): X may be phenylalanine 4
  mod_res (12)..(12): X may be proline
  mod_res (14)..(14): X may be threonine substituted with a polysaccharide
KEXEXLDXDFLXEX

SEQ ID NO: 10 - protein, 15 aa, Artificial Sequence
  Synthetic
  mod_res (3)..(3): X may be phenylalanine 4
  mod_res (5)..(5): X may be phenylalanine 4
  mod_res (8)..(8): X may be phenylalanine 4
  mod_res (12)..(12): X may be proline
  mod_res (14)..(14): X may be threonine substituted with a polysaccharide
KEXEXLDXDFLXEXE

SEQ ID NO: 11 - protein, 16 aa, Artificial Sequence
  Synthetic
  mod_res (3)..(3): X may be phenylalanine 4
  mod_res (5)..(5): X may be phenylalanine 4
  mod_res (8)..(8): X may be phenylalanine 4
  mod_res (12)..(12): X may be proline
  mod_res (14)..(14): X may be threonine substituted with a polysaccharide
KEXEXLDXDFLXEXEP

SEQ ID NO: 12 - protein, 17 aa, Artificial Sequence
  Synthetic
  mod_res (3)..(3): X may be phenylalanine 4
  mod_res (5)..(5): X may be phenylalanine 4
  mod_res (8)..(8): X may be phenylalanine 4
  mod_res (12)..(12): X may be proline
  mod_res (14)..(14): X may be threonine substituted with a polysaccharide
KEXEXLDXDFLXEXEPL